(19) United States
(12) Patent Application Publication
(10) Pub. No.: US 2026/0144871 A1
(43) Pub. Date: May 28, 2026

(54) Human Placental Derived Natural Killer Cells with High Affinity and Cleavage Resistant CD16 and Secreted IL-15, and uses thereof

(71) Applicant: Celularity Inc., Florham Park, NJ (US)

(72) Inventors: Lin KANG, Florham Park, NJ (US); Marina GERGUES, Florham Park, NJ (US); Yuechao ZHAO, Florham Park, NJ (US); Irene RAITMAN KHUTORSKOY, Florham Park, NJ (US); Shengchen Lin, Florham Park, NJ (US); Valentina ROUSSEVA, Florham Park, NJ (US); Shuyang HE, Florham Park, NJ (US); Adrian KILCOYNE, Florham park, NJ (US); Robert J. HARIRI, Florham Park, NJ (US)

(73) Assignee: Celularity Inc., Florham Park, NJ (US)

(21) Appl. No.: 18/921,811

(22) Filed: Oct. 21, 2024

Related U.S. Application Data
(60) Provisional application No. 63/629,417, filed on Oct. 20, 2023.

(51) Int. Cl. A61K 40/15; A61K 39/00; A61K 39/395; A61K 40/30; A61P 35/00

(52) U.S. Cl. CPC A61K 40/15; A61K 39/3955; A61K 40/30; A61P 35/00; A61K 2039/507

(57) ABSTRACT

Provided herein are new and useful placental derived natural killer (NK) Cell or population thereof, or one or more cells within the population that express a cleavage resistant CD 16 polypeptide and a secreted IL-15 polypeptide, as well as uses thereof.

### PRIORITY CLAIM

This application claims priority to U.S. Provisional Application 63/629,417, which is hereby incorporated by reference in its entirety.

### SEQUENCE LISTING INCORPORATION BY REFERENCE STATEMENT

Pursuant to 37 C.F.R. §§ 1.52 and 1. 834 (a) (2), Sequence Listing named “P1186USNPSEQLT.xml” consisting of 10,209 bytes, created on 18 Sep. 2025, is hereby incorporated by reference herein in its entirety.

### FIELD OF INVENTION

The present disclosure related to novel placental derived natural killer Cells that express a cleavage resistant CD16 and secret IL-15. Such cells of the instant disclosure readily have applications in treating numerous types of cancers.

### BACKGROUND

Natural killer (NK) cells exhibit innate anti-tumor activity owing to the expression of a multitude of activating and inhibitory receptors that orchestrate NK cell responses. It is thus possible to use NK cells from allogeneic sources without the risk of graft-vs-host disease, making them extremely attractive for developing “off-the-shelf” cellular therapies. Co-administration of such allogeneic NK cells and a cancer specific antigen antibody results the cells exhibiting Antibody Dependent Cellular Cytoxicity (ADCC). With this co-administration, such NK Cells can deleteriously affect cancer cells that come in contact with them.

In order to promote the survival, proliferation, and ADCC, the cytokine IL-15 is co-administered. This cytokine supports cell expansion and maintenance by: (a) inducing strong proliferative signals via JAK/STAT and Ras/MAPK signaling pathways, and (b) preventing cell death by increasing anti-apoptotic proteins Bcl-2 and Bcl-xL as well as decreasing pro-apoptotic proteins Bim and Puma through activation of PI3K pathway (Huntington N D, Puthalakath H, Gunn P, Naik E, Michalak E M, Smyth M J, et al., Interleukin 15-mediated survival of natural killer cells is determined by interactions among Bim, Noxa and Mcl-1, Nat Immunol. 2007; 8:856-63; Johnston J A, Bacon C M, Finbloom D S, Rees R C, Kaplan D, Shibuya K, et al., Tyrosine phosphorylation and activation of STAT5, STAT3, and Janus kinases by interleukins 2 and 15. Proc Natl Acad Sci USA. 1995; 92:8705-9; Miyazaki T, Kawahara A, Fujii H, Nakagawa Y, Minami Y, Liu Z J, et al., Functional activation of Jak1 and Jak3 by selective association with IL-2 receptor subunits. Science. 1994; 266:1045-7; Miyazaki T, Liu Z J, Kawahara A, Minami Y, Yamada K, Tsujimoto Y, et al., Three distinct IL-2 signaling pathways mediated by bcl-2, c-myc, and Ick cooperate in hematopoietic cell proliferation. Cell. 1995; 81:223-31; Adunyah S E, Wheeler B J, Cooper R S, Evidence for the involvement of LCK and MAP kinase (ERK-1) in the signal transduction mechanism of interleukin-15. Biochem Biophys Res Commun. 1997; 232:754-8; and Steelman L S, Pohnert S C, Shelton J G, Franklin R A, Bertrand F E, McCubrey J A, JAK/STAT, Raf/MEK/ERK, PI3K/Akt and BCR-ABL in cell cycle progression and leukemogenesis. Leukemia. 2004; 18:189-218). Additionally, IL-15 enhances the cytotoxic effector functions of lymphocytes by increasing production of perforin, a cytolytic pore forming protein, and death-inducing enzymes granzymes A/B, through all three pathways (Imada K, Bloom E T, Nakajima H, Horvath-Arcidiacono J A, Udy G B, Davey H W, et al., Stat5b is essential for natural killer cell-mediated proliferation and cytolytic activity. J Exp Med. 1998; 188:2067-74; Teglund S, Mckay C, Schuetz E, van Deursen J M, Stravopodis D, Wang D, et al., Stat5a and Stat5b proteins have essential and nonessential, or redundant, roles in cytokine responses. Cell. 1998; 93:841-50; and Farag S S, Caligiuri M A, Human natural killer cell development and biology. Blood Rev. 2006; 20:123-37).

What is needed though are NK cells that have superior anti-tumor activity, increased affinity for tumor cells, and increased availability than heretofore known NK Cells used in the treatment of cancer.

The citation of any reference herein should not be deemed as an admission that such reference is available as prior art to the instant disclosure.

### SUMMARY OF THE DISCLOSURE

Broadly, the instant disclosure extends to a placental derived natural killer (NK) cell or population thereof wherein the placental derived NK cell or one or more cells within the population of the placental derived NK Cell comprises:
a first polynucleotide encoding a cleavage resistant CD 16 polypeptide and a second polynucleotide encoding a secreted IL-15 polypeptide; or
a polynucleotide that encodes the cleavage resistant CD16 polypeptide and the secreted IL-15 polypeptide.

In a placental derived NK cell or population thereof the instant disclosure, the polynucleotide construct can further comprise a polynucleotide that encodes a self-cleaving peptide, such as, for example, the self-cleaving protein is a T2A peptide.

In a particular embodiment of a placental derived NK cell or population thereof of the instant disclosure, the polynucleotide construct encodes a polypeptide comprising the amino acid sequences of the cleavage resistant CD16, the T2A protein, and the secreted IL-15, wherein the cleavage resistant CD16 variant comprises a Valine residue at position 176 relative to the wild-type CD 16 polypeptide. Such a polynucleotide construct as well as a polypeptide construct that comprises the amino acid sequences of a cleavage resistant CD16, a T2A protein, and a secreted IL-15 are disclosed infra.

the present disclosure further extends to a placental derived natural killer (NK) Cell or population thereof, wherein the placental derived NK cell comprises, or one or more cells within the population of the placental derived NK Cell comprises (a) a first polynucleotide encoding a cleavage resistant CD 16 polypeptide; and (b) a second polynucleotide encoding a secreted IL-15 polypeptide, wherein the placental derived NK Cell or population thereof, or one or more cells of the population thereof, express the cleavage resistant CD16 polypeptide, and the secreted IL-15 polypeptide. In a particular embodiment of the present disclosure, a placental derived NK Cell or population thereof comprises a polynucleotide construct that comprises the first polynucleotide fused to a nucleotide sequence encoding a self-cleaving peptide, and the second polypeptide fused to the nucleotide sequence encoding the self-cleaving peptide. A particular embodiment of a nucleotide sequence encoding a self-cleaving site is a nucleotide sequence that encodes for a T2A peptide. Numerous variations of a construct as described herein have applications in a placental derived NK cell or population thereof of the instant disclosure. In a particular embodiment of the construct, the first polynucleotide sequence is 5′ of the nucleotide sequence encoding the self-cleaving peptide of the construct, and the second polynucleotide sequence construct is 3′ of the nucleotide sequence encoding the self-cleaving peptide.

The first polynucleotide of a cleavage resistant CD 16 can code a variety of such cleavage resistant CD 16s, including active isoforms thereof such as, for example, the CD 16a or CD16b isoforms. Moreover, the CD16b isoform can be further selected from the group consisting of an NA1 allelic variant and an NA2 allelic variant. In a particular embodiment, the cleavage resistant CD 16 variant may comprise:
(a) a Valine residue at position 176 relative to the wild-type CD 16 polypeptide;
(b) Ser, Pro Cys, Gly, Thr, or Phe at position 197 relative to the wild-type CD 16 polypeptide;
(c) Val or Leu at position 195 relative to the wild-type CD 16 polypeptide;
(d) any combination of (a) and (b).

Moreover, a cleavage resistant CD 16 variant having applications in a placental derived NK Cell or population thereof comprises an amino acid sequence identical to portions of other polypeptides, for example, the cleavage resistant CD 16 variant comprises an amino acid sequence:
(a) identical to a portion of a CD8 polypeptide;
(b) identical to the stalk region of CD8a;
(c) identical to a portion of a CD28 polypeptide;
(d) identical to the stalk region of a CD28 polypeptide;
(e) identical to a portion of a CD64 polypeptide; or
(f) identical to the stalk region of a CD64 polypeptide.
   In a particular embodiment, a cleavage resistant CD16 having applications herein comprises the amino acid sequence of:

(a)
[SEQ ID NO: 1]
TTTPAPRPPTPAPTIASQPLSLRPEACRPAAGGAVHTRGLDFACD
or
at least 15 consecutive residues of [SEQ ID NO: 1];
(b)
[SEQ ID NO: 2]
IEVMYPPPYLDNEKSNGTIIHVKGKHLCPSPLFPGPSKP,
or
at least 15 consecutive residues of [SEQ ID NO: 2];
(c)
[SEQ ID NO: 3]
PELELQVLGLQLPTPVWFH,
or
at least 15 consecutive amino acids of the sequence
PELELQVLGLQLPTPVWFH [SEQ ID NO: 3].

Moreover, a cleavage resistant CD 16 variant having applications herein can comprise an amino acid sequence of:

[SEQ ID NO: 4]
MWQLLLPTALLLLVSAGMRTEDLPKAVVFLEPQWYRVLEKDSVTLKCQG
AYSPEDNSTQWFHNESLISSQASSYFIDAATVDDSGEYRCQTNLSTLSD
PVQLEVHIGWLLLQAPRWVFKEEDPIHLRCHSWKNTALHKVTYLQNGKG
RKYFHHNSDFYIPKATLKDSGSYFCRGLVGSKNVSSETVNITITQGLAV
CTISSFFPPGYQVSFCLVMVLLFAVDTGLYFS VKTNIRS
STRDWKDHKFKWRKDPQDK.

placental derived NK cell or population thereof, or one or more cells within such a population of the instant disclosure comprise an amino acid tag at either the amino terminus or carboxy terminus. Particular examples of such tags having applications in the instant disclosure are 6×His (HHHHHH) [SEQ ID NO:5] tag or an myc tag EQKLISEEDL [SEQ ID NO: 6].

A placental derived NK cell or population thereof, or one or more cells within such a population of the instant disclosure can be characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPCI, MVD, CLK1, ARL4C, REL, KMT2E, YPEL5, AMD1, BTG2, and IDS that is less than the expression of the markers observed in peripheral blood natural killer cells. Moreover, expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 in a placental derived NK cell or population thereof, or one or more cells within such a population of the instant disclosure is greater than expression of the markers in observed peripheral blood natural killer cells.

The present disclosure further extends to a vector comprising (a) a first polynucleotide encoding a cleavage resistant CD 16 polypeptide operatively associated with a promoter and (b) a second polynucleotide encoding a secreted IL-15 polypeptide operatively associated with a promoter. Numerous types of vectors have applications herein. In a particular embodiment, the vector comprises a lentivirus vector. Naturally, one of ordinary skill in the art can readily convert a DNA sequence that expresses a cleavage resistant CD16 polypeptide and a secreted IL-15 into their respective RNA sequences for such a vector using routine laboratory techniques.

Moreover, a vector of the instant disclosure can comprise a polynucleotide construct as described herein operatively associated with a promoter, wherein the polynucleotide construct comprises the first polynucleotide fused to a nucleotide sequence encoding a self-cleaving peptide, and the second polypeptide fused to the nucleotide sequence encoding the self-cleaving peptide. As explained above, a particular embodiment of a polynucleotide construct as described herein, wherein the first polynucleotide sequence is 5′ of a nucleotide sequence encoding the self-cleaving peptide of the construct, and the second polynucleotide sequence of the construct is 3′ of the nucleotide sequence encoding the self-cleaving peptide.

In a particular embodiment, a polynucleotide construct as used herein encodes for an amino acid sequence that comprises a cleavage resistant CD16 variant, a T2A polypeptide and a secreted IL-14 (e.g. a CD16VS+T2A+IL-15 construct). Such a construct comprises the amino acid sequence of:

[SEQ ID NO: 7]
MWQLLLPTALLLLVSAGMRTEDLPKAVVFLEPQWYRVLEKDSVTLKCQG
AYSPEDNSTQWFHNESLISSQASSYFIDAATVDDSGEYRCQTNLSTLSD
PVQLEVHIGWLLLQAPRWVFKEEDPIHLRCHSWKNTALHKVTYLQNGKG
RKYFHHNSDFYIPKATLKDSGSYFCRGLVGSKNVSSETVNITITQGVIT
ALSSSFFPPGYQVSFCLVMVLLFAVDTGLYFSVKTNIRSSTRDWKDHKF
KWRKDPQDKGSGEGRGSLLTCGDVEENPGPMRISKPHLRSISIQCYLCL
LLNSHFLTEAGIHVFILGCFSAGLPKTEANWVNVISDLKKIEDLIQSMH
IDATLYTESDVHPSCKVTAMKCFLLELQVISLESGDASIHDTVENLIIL
ANNSLSSNGNVTESGCKECEELEEKNIKEFLQSFVHIVQMFINT.

Due to degeneracy numerous polynucleotide constructs, including those described above, have applications in encoding the CD16VS+T2A+IL-15 polypeptide construct of [SEQ ID NO: 5]. A particular example of such a polynucleotide construct comprise the nucleotide sequence of [SEQ ID NO:8]:

[SEQ ID NO: 8]
ATGTGGCAGCTGCTCCTCCCAACTGCTCTGCTACTTCTAGTTTCAGCTG
GCATGCGGACCGAGGACCTGCCCAAGGCCGTGGTGTTCCTGGAGCCCCA
GTGGTACCGGGTGCTGGAGAAGGACAGCGTGACCCTGAAGTGCCAGGGC
GCCTACAGCCCCGAGGACAACAGCACCCAGTGGTTCCACAACGAGAGCC
TGATCAGCAGCCAGGCCAGCAGCTACTTCATCGACGCCGCCACCGTGGA
CGACAGCGGCGAGTACCGGTGCCAGACCAACCTGAGCACCCTGAGCGAC
CCCGTGCAGCTGGAGGTGCACATCGGCTGGCTGCTGCTGCAGGCCCCCC
GGTGGGTGTTCAAGGAGGAGGACCCCATCCACCTGCGGTGCCACAGCTG
GAAGAACACCGCCCTGCACAAGGTGACCTACCTGCAGAACGGCAAGGGC
CGGAAGTACTTCCACCACAACAGCGACTTCTACATCCCCAAGGCCACCC
TGAAGGACAGCGGCAGCTACTTCTGCCGGGGCCTGGTGGGCAGCAAGAA
CGTGAGCAGCGAGACCGTGAACATCACCATCACCCAGGGCGTGATCACC
GCCCTGAGCAGCAGCTTCTTCCCCCCCGGCTACCAGGTGAGCTTCTGCC
TGGTGATGGTGCTGCTGTTCGCCGTGGACACCGGCCTGTACTTCAGCGT
GAAGACCAACATCCGGAGCAGCACCCGGGACTGGAAGGACCACAAGTTC
AAGTGGCGGAAGGACCCCCAGGACAAGGGAAGTGGTGAAGGACGGGGAT
CACTGCTCACTTGCGGGGACGTCGAAGAGAACCCAGGCCCAATGCGAAT
TTCTAAGCCTCACCTCCGAAGTATTAGCATACAGTGCTATCTGTGTCTT
TTGCTGAACTCTCACTTTCTGACCGAAGCGGGAATCCATGTATTCATCC
TCGGGTGTTTTTCAGCGGGCCTCCCAAAGACAGAGGCGAACTGGGTCAA
CGTCATATCTGACCTCAAGAAAATTGAGGATTTGATACAATCAATGCAT
ATTGATGCGACACTCTACACTGAGAGCGATGTTCACCCAAGCTGTAAGG
TAACGGCTATGAAATGTTTTCTCTTGGAGCTCCAGGTTATTTCATTGGA
GTCAGGGGACGCAAGTATACACGACACTGTGGAAAATCTTATAATCCTG
GCTAATAACTCCCTCAGTTCTAATGGAAATGTCACTGAGTCTGGATGCA
AAGAGTGCGAAGAGTTGGAAGAGAAGAATATCAAAGAGTTTCTTCAGTC
CTTCGTGCATATAGTCCAAATGTTTATCAACACCTCCTGATAA,

or a degenerate variant thereof.

In particular embodiment, a polynucleotide construct of a vector of the instant

disclosure encodes a polypeptide comprising the
amino acid sequece of:
[SEQ ID NO: 7]
MWQLLLPTALLLLVSAGMRTEDLPKAVVFLEPQWYRVLEKDSVTLKCQG
AYSPEDNSTQWFHNESLISSQASSYFIDAATVDDSGEYRCQTNLSTLSD
PVQLEVHIGWLLLQAPRWVFKEEDPIHLRCHSWKNTALHKVTYLQNGKG
RKYFHHNSDFYIPKATLKDSGSYFCRGLVGSKNVSSETVNITITQGVIT
ALSSSFFPPGYQVSFCLVMVLLFAVDTGLYFSVKTNIRSSTRDWKDHKF
KWRKDPQDKGSGEGRGSLLTCGDVEENPGPMRISKPHLRSISIQCYLCL
LLNSHFLTEAGIHVFILGCFSAGLPKTEANWVNVISDLKKIEDLIQSMH
IDATLYTESDVHPSCKVTAMKCFLLELQVISLESGDASIHDTVENLIIL
ANNSLSSNGNVTESGCKECEELEEKNIKEFLQSFVHIVQMFINT.

Moreover, the present disclosure extends to a placental derived NK cell or population thereof, or one or more cells of such population as disclosed herein, transformed or transfected with a vector of the instant disclosure.

Various Cell subtypes have applications in a placental derived NK Cell of the instant disclosure, a population thereof, or one or more cells of such a population as disclosed herein. For example, in particular embodiments:
(i) greater than 90% of the cells in the population are CD56+ and CD3−;
(ii) less than 1% of the cells in the population are CD3+;
(iii) less than 1% of the cells in the population are CD19+;
(iv) greater than 65% of the cells in the population are CD16+; or
(v) any combination of (i)-(iv).

A placental derived NK cell or population thereof, or one or more cells of such population of the instant disclosure can be characterized in a number of ways. For example, such cells or population thereof or one or more cells of such a population of the instant disclosure:
(a) expresses one or more surface markers selected from the group consisting of CD226, NKG2D, CD 11 a, NKp30, NKp44, NKp46, CD94, and combinations thereof;
(b) exhibits greater antibody-dependent cellular cytotoxicity than that observed in a population of placental-derived natural killer T-cells lacking expression of the cleavage resistant CD 16.
(c) exhibits greater vitality and thus, increased activity as compared to that observed in a population of placental derived NK Cells, or one or more cells thereof, which do not express IL-15; or
(d) any combination of (a)-(d).

The present disclosure further extends to a method of suppressing the proliferation of tumor cells comprising contacting the tumor cells with:
(i) a placental derived natural killer cell or population thereof or one or more cells of population thereof of the instant disclosure, wherein the cell or one or more cells within the population thereof comprises:
   (a) a first polynucleotide encoding a cleavage resistant CD 16 polypeptide; and
   (b) a second polynucleotide encoding a secreted IL-15 polypeptide; and
(ii) an antibody against a cancer specific antigen.

Optionally, a placental derived NK cell or population of cells as disclosed herein are CYNK cells, and particularly placental CD34+ cells. Moreover, numerous antibodies having a cancer specific antigen have applications in the instant disclosure, including an antibody having HER2 as its antigen, e.g. trastuzumab (HERCEPTIN), or an antibody having CD20 has its antigen, e.g. rituximab (RITUXAN). A cancer specific antigen antibody having applications herein can be monoclonal, polyclonal, fully human, humanized, chimeric or any combination thereof.

Contacting a placental derived NK Cell, or population thereof, or one or more cells of such population, that expresses a cleavage resistant CD16 polypeptide and a secreted IL-15 polypeptide can occur in vitro, in vivo, or in a human.

The tumor cells of a method or use of a composition of the present disclosure can be from numerous types of cancers, including Bladder cancers, Breast cancers, Cervical cancers, Cholangiocarcinomas (extrahepatic), Cholangiocarcinomas (intrahepatic), Colorectal cancers, Esophageal or esophagogastric junction cancers, Gallbladder cancers, Gastric adenocarcinomas, Gastrointestinal stromal tumors, Glioblastoma multiforme, high grade gliomas, Gliomas (low grade), Head and neck carcinomas, Hepatocellular carcinomas, Intestinal (small) malignancies, lymphoma malignancies, Kidney cancers, Lung cancers (non-small cells), Lung cancers (small cells), Melanomas, Melanomas (uveal), Neuroendocrine tumors, Oligodendrogliomas, Ovarian (epithelial) cancers, Ovarian (non-epithelial) cancers, Pancreatic adenocarcinomas, Penile cancers, Pituitary cancers, Prostate cancers, Sarcomas (peritoneal, retroperitoneal), Sarcomas (soft tissues), Solitary fibrous tumors, Testicular cancers, Thymic cancers, Thyroid cancers, Uterine cancers, and combinations thereof.

Particular tumor cells having applications herein are gastric cancer cells, e.g., HER2+ gastric cancer cells NCI-N87, or CD20+ Burkitts lymphoma Daudi cells.

Also provided herein is use of a composition comprising a placental derived NK cell or population of human placental-derived natural killer cells that express a cleavage resistant CD 16 and a secreted IL15 for suppressing the proliferation of tumor cells.

These and other aspects of the present disclosure will be better appreciated by reference to the following drawings and Detailed Description.

### BRIEF DESCRIPTION OF THE DRAWINGS

FIG. 1. (A) Percent CD56+CD3− on non-transduced CYNK (CYNK-NT) and CYNK-201 post-thaw (n=5 donors). (B) Fold expansion after 35 days in culture for CYNK-NT and CYNK-201 with comparable cell yield (n=5 donors). (C) Post-thaw phenotyping for NK surface markers CD226, NKG2D, CD94, CD11a, NKp30, NKp44 and NKp46 gated under the CD56+CD3− population for CYNK-NT and CYNK-201 with comparable expression patterns except where indicated, *p<0.05, **p<0.01 (n=5 donors).

FIG. 2. (A) CD16 expression on CD56+CD3− CYNK post-thaw (n=5 donors, ****p<0.0005). (B) Quantification of CD16 cleavage on CYNK cells (n=5 donors, ****p<0.0005). (C) Representative flow cytometry diagrams showing CD16 loss following cleavage of CYNK-NT but resistance on CYNK-201 following activation by PMA/ionomycin (PMAi). (D) IL-15 secretion of CYNK-201 (n=5 donors) was confirmed by ELISA.

FIG. 3. 4 hr and 24 hr ADCC of CYNK in combination with trastuzumab compared to the IgG control antibody against HER2+ gastric cancer cell line NCI-N87 at the indicated E:T ratios (n=5 donors, *p<0.05, **p<0.01, ***p<0.005 for CYNK-201 w/Tras vs. CYNK-NT w/Tras).

FIG. 4. 4 hr and 24 hr ADCC of CYNK in combination with rituximab compared to the IgG control antibody against CD20⁺ B-cell lymphoma cell line Daudi at the indicated E:T ratios (n=5 donors, *p<0.05, **p<0.01, ***p<0.005 for CYNK-201 w/Ritux vs. CYNK-NT w/Ritux).

FIG. 5. Level of GM-CSF, IFN-γ and TNF-α (ng/1×10⁶ cells) following 24 hr co-culture with NCI-N87 at 1:1 E:T ratio in combination with IgG control or with trastuzumab, or following 24 h stimulation with PMAi as a positive control (n=5 donors, *p<0.05 for CYNK-201 w Tras vs. CYNK-201 w IgG and vs. CYNK-201 alone).

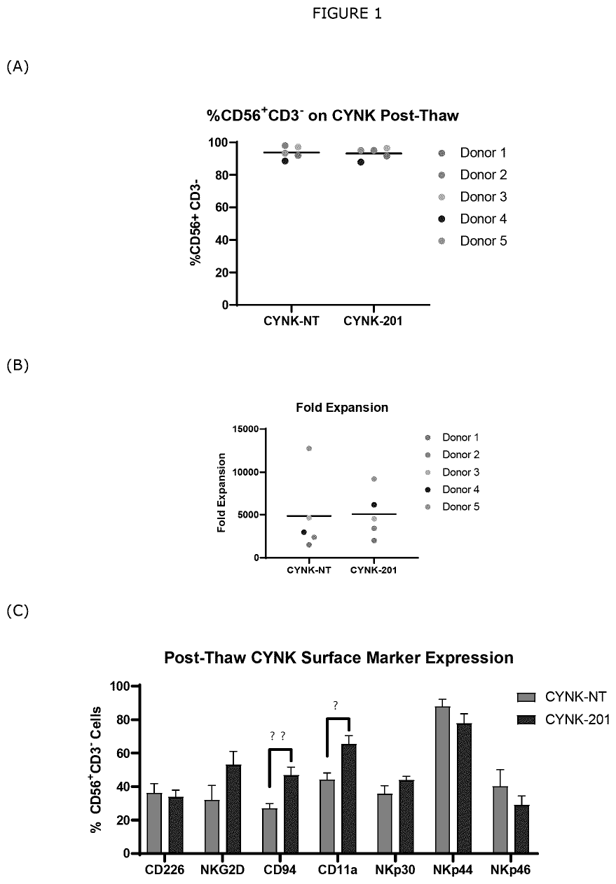

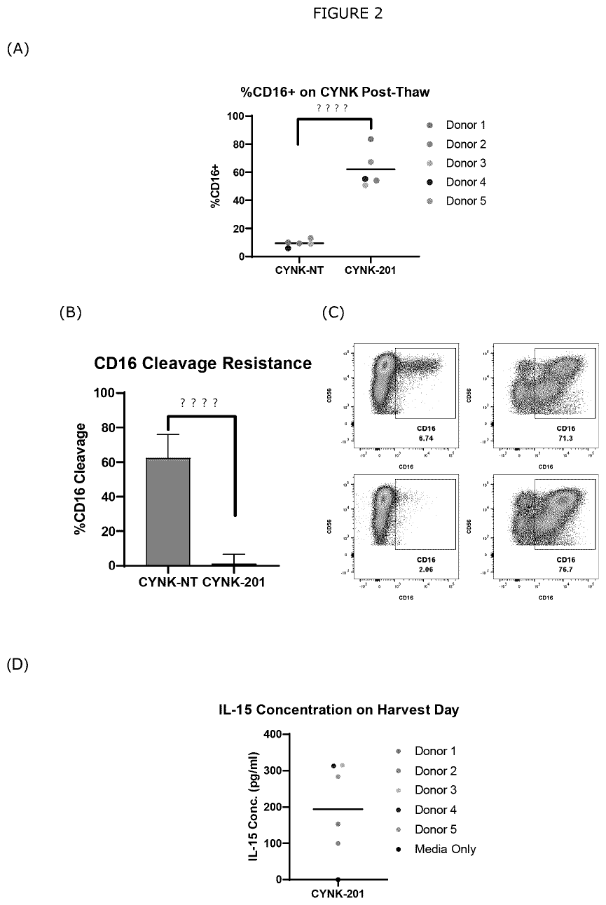

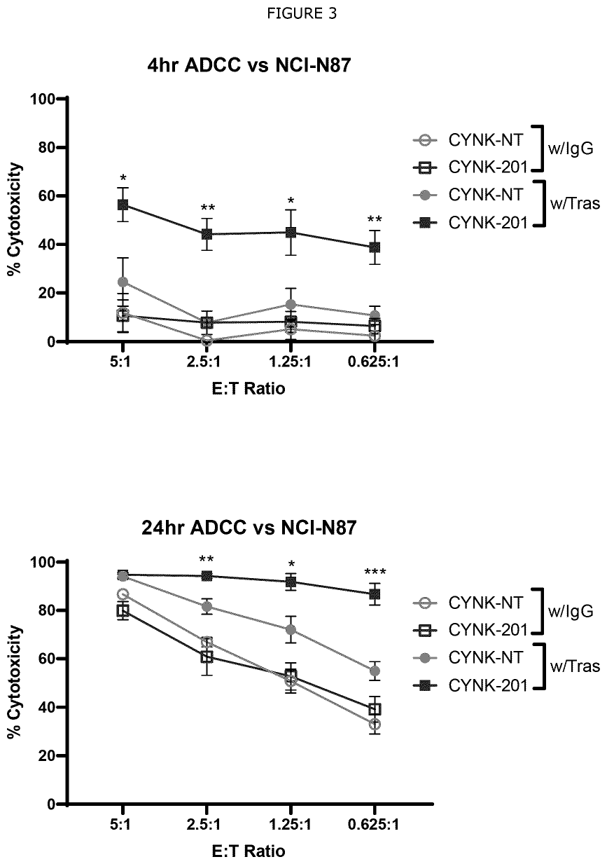

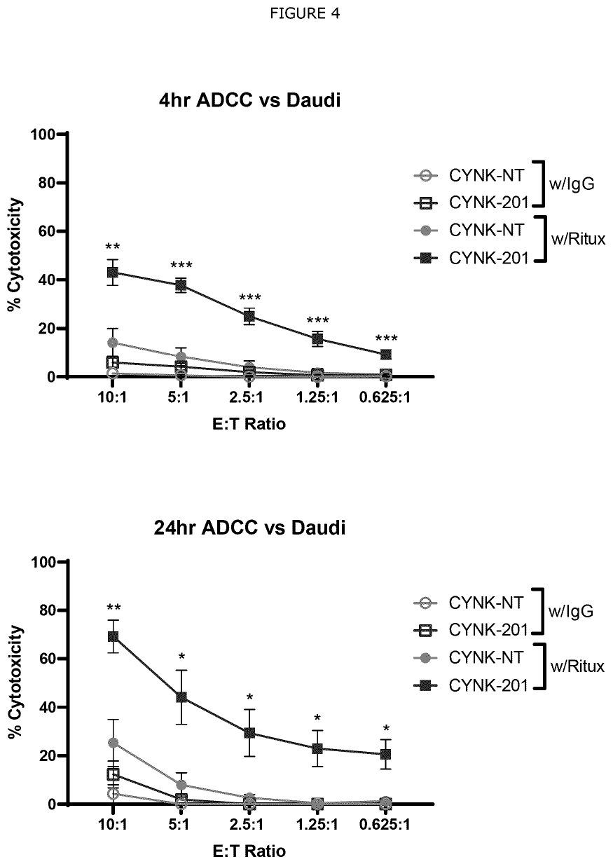

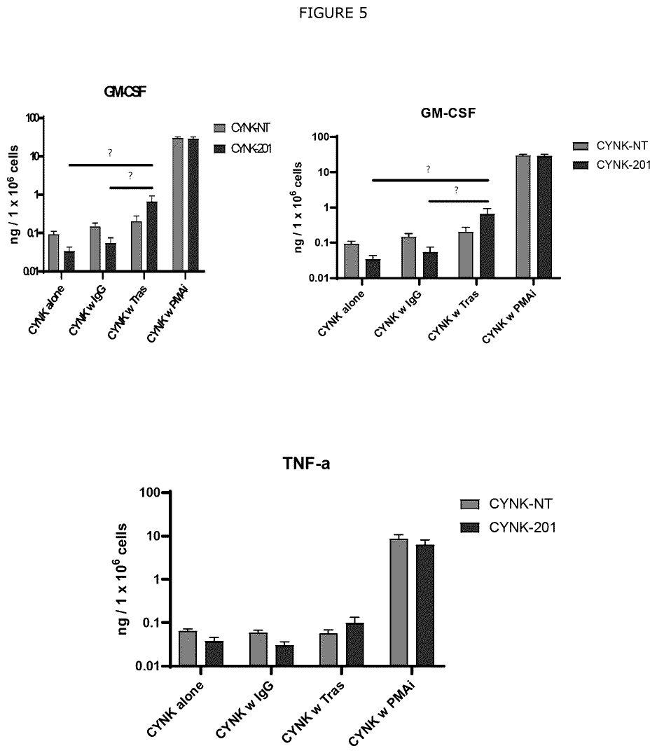

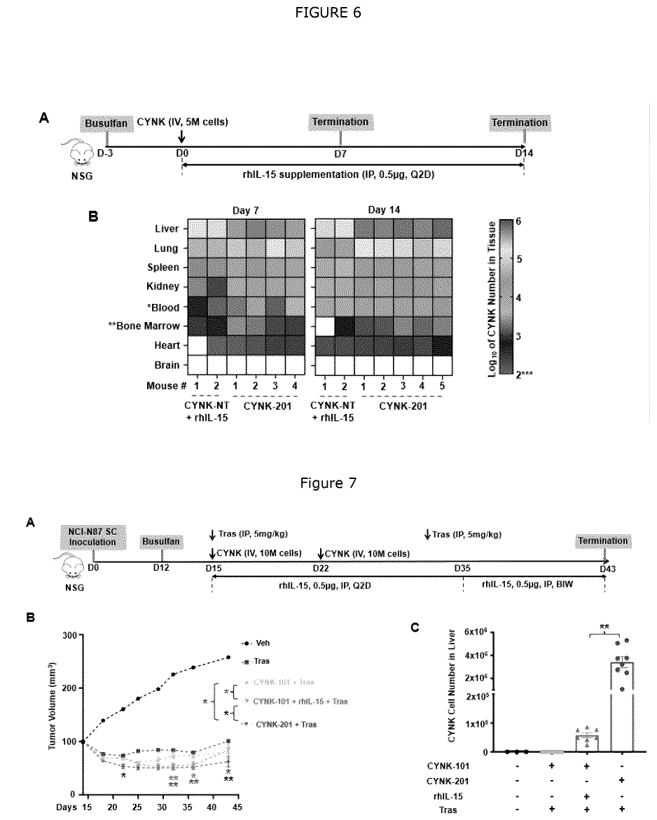

FIG. 6. (A) In vivo persistence & biodistribution study scheme: 3 days after lymphodepletion by busulfan, NSG mice were IV injected with CYNK cells supplemented with or without recombinant human IL-15 (rhIL-15). On Day 7 or 14, mice were sacrificed to analyze CYNK cell persistence & biodistribution by digital PCR (dPCR). (B) CYNK cell numbers were calculated to Log₁₀ values and presented as heatmap based on dPCR results of indicated whole organs or tissues.
CYNK cell number of each mouse was calculated from 50-100 μL blood sample×estimated whole blood volume (80 μL per gram of mouse body weight).
*CYNK cell number in bone marrow was estimated by CYNK cell number calculated from each femur×4.
*Tissue samples for which dPCR data was lower than cutoff value (2) are presented in white color.

FIG. 7. (A) In vivo anti-tumor efficacy study scheme: NSG mice were inoculated subcutaneously with NCI-N87 cells (Day 0), given busulfan (Day 12), treated with vehicle, trastuzumab (Tras) alone, or Tras plus two injections of CYNK-201 or CYNK cells expressing high affinity and cleavage resistant CD16 only (CYNK-101), then supplemented with or without rhIL-15. (B) Tumor volumes were measured and presented. Two-way ANOVA with Dunnett's multiple comparisons test (n=8-10 animals, *p<0.05, **p<0.01) were performed among indicated comparisons. (C) CYNK cell number was calculated and compared based on dPCR analysis of whole livers collected from mice by the end of study (Day 43) for T-test comparison (n=8 animals, **p<0.01).

### DETAILED DESCRIPTION

Broadly, the present disclosure extends to a placental derived natural killer (NK) Cell or population thereof, wherein the placental derived NK cell comprises, or one or more cells within the population of the placental derived NK Cell comprises (a) a first polynucleotide encoding a cleavage resistant CD 16 polypeptide; and (b) a second polynucleotide encoding a secreted IL-15 polypeptide, wherein the placental derived NK Cell or population thereof, or one or more cells of the population thereof, express the cleavage resistant CD16 polypeptide, and the secreted IL-15 polypeptide. In a particular embodiment of the present disclosure, a placental derived NK Cell or population thereof comprises a polynucleotide construct that comprises the first polynucleotide fused to a nucleotide sequence encoding a self-cleaving peptide, and the second polypeptide fused to the nucleotide sequence encoding the self-cleaving peptide.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure.

All numerical designations, e.g. volume, mass, number of resin particles, etc. are approximations which are varied by (+) or (−) by increments of 1.0 or 0.1, as appropriate. It is to be understood, although not always explicitly stated, that all numerical designations are preceded by the term “about”.

Numerous terms and phrases are used throughout the instant specification and claims and are defined below.

“About” and “approximately” are interchangeable and mean plus or minus a percent (e.g. +5%) of the number, parameter, or characteristic so qualified, which would be understood as appropriate by a skilled artisan to the scientific context in which the term is utilized.

As used here, the singular form “a”, “an” and “the” include plural reference unless the context clearly dictates otherwise.

As used herein, the terms “comprising”, “comprises” and “comprise” are intended to mean that the compositions, preparations and methods disclosed herein include recited elements, but do not exclude others.

As used herein Therefore, if appearing herein, the following terms shall have the definitions set out below.

A “vector” is a replicon, such as plasmid, phage, virus or cosmid, to which another DNA segment may be attached so as to bring about the replication of the attached segment. A “replicon” is any genetic element (e.g., plasmid, chromosome, virus) that functions as an autonomous unit of DNA replication in vivo, i. e., capable of replication under its own control In a particular embodiment of the instant disclosure, the vector is a lentivirus.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation. Thus, in the instant disclosure, the cassette can comprise the first polynucleotide and the second polynucleotide, or a polynucleotide construct as described above.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Heterologous” DNA refers to DNA not naturally located in the cell, or in a chromosomal site of the cell. Optionally, the heterologous DNA includes a gene foreign to the cell.

A “nucleic acid molecule” or “nucleotide sequence”, or “polynucleotide” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i. e., the strand having a sequence homologous to the mRNA). A “recombinant DNA molecule” is a DNA molecule that has undergone a molecular biological manipulation.

A “promoter sequence” or “promoter” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease SI), as well as protein binding domains (consensus sequences) responsible for the binding of RNA polymerase.

A coding sequence is “operatively associated” with transcriptional and translational control sequences in a cell when RNA polymerase transcribes the coding sequence into mRNA, which is then trans-RNA spliced and translated into the protein encoded by the coding sequence.

Moreover, due to the degenerate nature of codons in the genetic code, a polypeptide as described above of the present disclosure can be encoded by numerous isolated nucleic acid molecules. “Degenerate nature” refers to the use of different three-letter codons to specify a particular amino acid pursuant to the genetic code. It is well known in the art that the following codons can be used interchangeably to code for each specific amino acid: Phenylalanine (Phe or F) UUU or UUC Leucine (Leu or L) UUA or UUG or CUU or CUC or CUA or CUG Isoleucine (He or I) AUU or AUC or AUA Methionine (Met or M) AUG Valine (Val or V) GUU or GUC of GUA or GUG Serine (Ser or S) UCU or UCC or UCA or UCG or AGU or AGC.

Proline (Pro or P) CCU or CCC or CCA or CCG Threonine (Thr or T) ACU or ACC or ACA or ACG Alanine (Ala or A) GCU or GCG or GCA or GCG Tyrosine (Tyr or Y) UAU or UAC Histidine (His or H) CAU or CAC Glutamine (Gin or Q) CAA or CAG Asparagine (Asn or N) AAU or AAC Lysine (Lys or K) AAA or AAG Aspartic Acid (Asp or D) GAU or GAC Glutamic Acid (Glu or E) GAA or GAG Cysteine (Cys or C) UGU or UGC Arginine (Arg or R) CGU or CGC or CGA or CGG or AGA or AGG Glycine (Gly or G) GGU or GGC or GGA or GGG Tryptophan (Trp or W) UGG Termination codon UAA (ochre) or UAG (amber) or UGA (opal) It should be understood that the codons specified above are for RNA sequences. The corresponding codons for DNA have a T substituted for U.

Isolated nucleic acid molecules of the present disclosure, including polynucleotide constructs, can be inserted into an appropriate expression vector, i.e., a vector which contains the necessary elements for the transcription and translation of the inserted protein-coding sequence. Such elements are termed herein a “promoter.” Thus, a first polynucleotide encoding a cleavage resistant CD 16 polypeptide, a second polynucleotide encoding a secreted IL-15 polypeptide, and a polynucleotide construct of the instant disclosure, along with isolated nucleic acid molecules hybridizable thereto under standard hybridization conditions, operatively associated with a promoter can be inserted into an expression vector of the invention. A DNA sequence is “operatively associated” to an expression control sequence, such as a promoter, when the expression control sequence controls and regulates the transcription and translation of that DNA sequence. The term “operatively associated” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If an isolated nucleic acid molecule of the present invention does not contain an appropriate start signal, such a start signal can be inserted into the expression vector in front of (5′ of) the isolated nucleic acid molecule. Both cDNA and genomic sequences can be cloned and expressed under control of such regulatory sequences. An expression vector also preferably includes a replication origin. Hence, expression of the CD16SV+T2A+IL-15 can readily expressed in a recombinant expression vector comprising the nucleotide sequence of SEQ ID NO: 8 operatively associated with a promoter. The necessary transcriptional and translational signals can be provided on such a recombinant expression vector.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; viral vectors, bacteria transformed with bacteriophage, DNA, plasmid DNA, or cosmid DNA. In a particular embodiment, the expression vector is a lentivirus vector. The expression elements of vectors vary in their strengths and specificities. Depending on the host-vector system utilized, any one of a number of suitable transcription and translation elements may be used.

Moreover, an isolated nucleic acid molecule of the present invention may be expressed chromosomally, after integration of the coding sequence by recombination. In this regard, any of a number of amplification systems may be used to achieve high levels of stable gene expression (See Sambrook et al, 1989, supra).

A unicellular host containing a recombinant vector comprising an isolated nucleic acid molecule, or degenerate variants thereof, or an isolated nucleic acid molecule hybridizable under standard hybridization conditions to an isolated nucleic acid molecule, or degenerate variants thereof, such as the first polynucleotide, the second polynucleotide, and the polynucleotide construct of the present disclosure, which cleavage resistant CD 16 polypeptide, a second polynucleotide encoding a secreted IL-15 polypeptide, is cultured in an appropriate cell culture medium under conditions that provide for expression of the isolated nucleic acid molecule by the cell.

Any of the methods previously described for the insertion of DNA fragments into a cloning vector may be used to construct expression vectors comprising an isolated nucleic acid molecule of the present disclosure, and appropriate transcriptional/translational control signals and the protein coding sequences. These methods may include in vitro recombinant DNA and synthetic techniques and in vivo recombination (genetic recombination). Expression of an isolated nucleic acid molecule of the present invention, degenerate variants thereof, or an isolated nucleic acid molecule hybridizable thereto under standard hybridization conditions, along with polynucleotide construct, encoding a cleavage resistant CD 16 polypeptide and a secreted IL-15 polypeptide, degenerate variants thereof, or an isolated nucleic acid molecule hybridizable thereto under standard hybridization conditions, may be controlled by any promoter/enhancer element known in the art, but these regulatory elements must be functional in the host selected for expression. Promoters which may be used to control expression include, but are not limited to, the SV40 early promoter region (Benoist and Chambon, 1981, Nature 290:304-310), the promoter contained in the 3′ long terminal repeat of Rous sarcoma virus (Yamamoto, et al, 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the toe promoter (DeBoer, et al., 1983, Proc. Natl. Acad. Sci. U.S.A. 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al, 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al, 1987, Genes and Devel. 1:161-171), beta-globin gene control region which is active in myeloid cells (Mogram et al., 1985, Nature 315:338-340; Kollias et al., 1986, Cell 46:89-94), myelin basic protein gene control region which is active in oligodendrocyte cells in the brain (Readhead et al., 1987, Cell 48:703-712), myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

Expression vectors comprising an isolated nucleic acid molecule, or degenerate variants thereof, encoding a cleavage resistant CD 16 polypeptide, and a secreted IL-15 polypeptide, or an expression vector comprising an isolated nucleic acid molecule hybridizable under standard hybridization conditions to an isolated nucleic acid molecule described herein, can be identified by four general approaches: (a) PCR amplification of the desired plasmid DNA or specific mRNA, (b) nucleic acid hybridization, (c) presence or absence of selection marker gene functions, and (d) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if an isolated nucleic of the present invention, or degenerate variants thereof, which encode a cleavage resistant CD 16 polypeptide and a secreted IL-15 polypeptide, including a construct of the present disclosure (e.g. a polynucleotide construct of the present disclosure), or an isolated nucleic acid molecule hybridizable thereto under standard hybridization conditions, is inserted within the “selection marker” gene sequence of the vector, recombinants containing the insert can be identified by the absence of the inserted gene function. In the fourth approach, recombinant expression vectors can be identified by assaying for the activity, biochemical, or immunological characteristics of the gene product expressed by the recombinant, provided that the expressed protein assumes a functionally active conformation.

### Production of a Cleavage Resistant CD 16 Polypeptide and a Secreted IL-15 Polypeptide Hematopoietic Cells

Hematopoietic cells useful in the instant disclosure are disclosed in PCT Published Patent Application WO2023137344A1, which is hereby incorporated by reference in its entirety. Any hematopoietic cells able to differentiate into NK cells having applications herein. Hematopoietic cells can be obtained from tissue sources such as, e.g., bone marrow, cord blood, placental blood, peripheral blood, liver or the like, or combinations thereof. Hematopoietic cells can be obtained from placenta. In a specific embodiment, the hematopoietic cells are obtained from placental perfusate. In one embodiment, the hematopoietic cells are not obtained from umbilical cord blood. In one embodiment, the hematopoietic cells are not obtained from peripheral blood. Hematopoietic cells from placental perfusate can comprise a mixture of fetal and maternal hematopoietic cells, e.g., a mixture in which maternal cells comprise greater than 5% of the total number of hematopoietic cells. In certain embodiments, hematopoietic cells from placental perfusate comprise at least about 90%, 95%, 98%, 99% or 99.5% fetal cells.

In an embodiment of the instant disclosure, the hematopoietic cells, e.g., hematopoietic stem cells or progenitor cells, from which the NK cell or population thereof of the instant disclosure can be combined cells from placental perfusate and cord blood, e.g., cord blood from the same placenta as the perfusate or different placentas. In another specific embodiment, said umbilical cord blood is isolated from a placenta other than the placenta from which said placental perfusate is obtained. In certain embodiments, the combined cells can be obtained by pooling or combining the cord blood and placental perfusate. In certain embodiments, the cord blood and placental perfusate are combined at a ratio of 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by volume to obtain the combined cells. In a specific embodiment, the cord blood and placental perfusate are combined at a ratio of from 10:1 to 1:10, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10. In a more specific embodiment, the cord blood and placental perfusate are combined at a ratio of 8.5:1.5 (85%: 15%).

In certain embodiments, the cord blood and placental perfusate are combined at a ratio of 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by total nucleated cells (TNC) content to obtain the combined cells. In a specific embodiment, the cord blood and placental perfusate are combined at a ratio of from 10:1 to 10:1, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10.

In another specific embodiment, the hematopoietic cells, e.g., hematopoietic stem cells or progenitor cells from which a said NK cell populations can be produced using a three-stage method described herein are produced, can be from both umbilical cord blood and placental perfusate, but wherein said umbilical cord blood is isolated from a placenta other than the placenta from which said placental perfusate is obtained.

In certain embodiments, the hematopoietic cells are CD34+ cells. In specific embodiments, the hematopoietic cells useful in the methods disclosed herein are CD34+CD38+ or CD34+CD38. In a more specific embodiment, the hematopoietic cells are CD34 CD38 Lin′. In another specific embodiment, the hematopoietic cells are one or more of CD2. CD3. CD lib, CD lie, CD 14, CD 16, CD 19, CD24, CD56, CD66b and/or glycophorin A″. In another specific embodiment, the hematopoietic cells are CD2. CD3. CDIIb′, CDIIc″, CD14″, CD16, CD19, CD24, CD56, CD66b and glycophorin A″. In another more specific embodiment, the hematopoietic cells are CD34 CD38 CD33 CD I 17. In another more specific embodiment, the hematopoietic cells are CD34 CD38 CD33 CDI 17 CD235 CD36.

In another embodiment, the hematopoietic cells are CD45+. In another specific embodiment, the hematopoietic cells are CD34+CD45+. In another embodiment, the hematopoietic cell is Thy−1+. In a specific embodiment, the hematopoietic cell is CD34+Thy−1+. In another embodiment, the hematopoietic cells are CD133+. In specific embodiments, the hematopoietic cells are CD34+CD133+ or CD133+Thy−1+. In another specific embodiment, the CD34+ hematopoietic cells are CXCR4+. In another specific embodiment, the CD34+ hematopoietic cells are CXCR4. In another embodiment, the hematopoietic cells are positive for KDR (vascular growth factor receptor 2). In specific embodiments, the hematopoietic cells are CD34+KDR+, CD133+KDR+ or Thy−1+KDR+. In certain other embodiments, the hematopoietic cells are positive for aldehyde dehydrogenase (ALDH+), e.g., the cells are CD34+ALDH+.

In certain other embodiments, the CD34+ cells are CD45. In specific embodiments, the CD34+ cells, e.g., CD34+, CD45 cells express one or more, or all, of the miRNAs hsa-miR-380, hsa-miR-512, hsa-miR-517, hsa-miR-518c, hsa-miR-519b, hsa-miR-520a, hsa-miR-337, hsa-miR-422a, hsa-miR-549, and/or hsa-miR-618.

In certain embodiments, the hematopoietic cells are CD34.

The hematopoietic cells can also lack certain markers that indicate lineage commitment, or a lack of developmental naivete. For example, in another embodiment, the hematopoietic cells are HLA-DR. In specific embodiments, the hematopoietic cells are CD34+HLA-DR, CD133+HLA-DR, Thy−1+HLA-DR or ALDH+HLA-DR In another embodiment, the hematopoietic cells are negative for one or more, or all, of lineage markers CD2, CD3, CDIlb, CDI Ic, CD14, CD16, CD19, CD24, CD56, CD66b and glycophorin A. Thus, hematopoietic cells can be selected for use in the methods disclosed herein on the basis of the presence of markers that indicate an undifferentiated state, or on the basis of the absence of lineage markers indicating that at least some lineage differentiation has taken place. Methods of isolating cells, including hematopoietic cells, on the basis of the presence or absence of specific markers is discussed in detail below.

### Placental Hematopoietic Stem Cells

In certain embodiments, the hematopoietic cells used in the methods provided herein are placental hematopoietic cells. In one embodiment, placental hematopoietic cells are CD34+. In a specific embodiment, the placental hematopoietic cells are predominantly (e.g., at least about 50%, 55%, 60%, 65%, 70%, 75%, 80%, 85%, 90%, 95% or 98%) CD34 CD38 cells. In another specific embodiment, the placental hematopoietic cells are predominantly (e.g., at least about 50%, 55%, 60%, 65%, 70%, 75%, 80%, 85%, 90%, 95% or 98%) CD34+CD38+ cells. Placental hematopoietic cells can be obtained from a postpartum mammalian (e.g., human) placenta by any means known to those of skill in the art, e.g., by perfusion.

In another embodiment, the placental hematopoietic cell is CD45. In a specific embodiment, the hematopoietic cell is CD34+CD45. In another specific embodiment, the placental hematopoietic cells are CD34+CD45+.

5.2. Production of Natural Killer and/or ILC3 Cells and Natural Killer Cell and/or ILC3 Cell Populations

Production of placenta derived NK cells populations thereof comprises expanding a population of hematopoietic cells. During cell expansion, a plurality of hematopoietic cells within the hematopoietic cell population differentiate into NK cells. In one aspect, provided herein is a method of producing NK cells comprising culturing hematopoietic stem cells or progenitor cells, e.g., CD34+ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56+, CD3−, and wherein at least 70%, for example at least 80%, of the natural killer cells are viable. In certain embodiments, such natural killer cells comprise natural killer cells that are CD16−. In certain embodiments, such natural killer cells comprise natural killer cells that are CD94+. In certain embodiments, such natural killer cells comprise natural killer cells that are CD94+ or CD16+. In certain embodiments, such natural killer cells comprise natural killer cells that are CD94− or CD16−. In certain embodiments, such natural killer cells comprise natural killer cells that are CD94+ and CD16+. In certain embodiments, such natural killer cells comprise natural killer cells that are CD94− and CD16−. In certain embodiments, said first medium and/or said second medium lack leukemia inhibiting factor (LIF) and/or macrophage inflammatory protein-1 alpha (MIP-la). In certain embodiments, said third medium lacks LIF, MIP-la, and FMS-like tyrosine kinase-3 ligand (FIt-3L). In specific embodiments, said first medium and said second medium lack LIF and MIP-la, and said third medium lacks LIF, MIP-la, and Fit3L. In certain embodiments, none of the first medium, second medium or third medium comprises heparin, e.g., low-molecular weight heparin.

### Production of a Placental Derived NK Cell or Population Thereof Using a Three-Stage Method

In one embodiment, provided herein is a three-stage method of producing NK cell populations. In certain embodiments, the method of expansion and differentiation of the hematopoietic cells, as described herein, to produce NK cell populations according to a three-stage method described herein comprises maintaining the cell population comprising said hematopoietic cells at between about 2×10⁴ and about 6×10⁶ cells per milliliter. In certain aspects, said hematopoietic stem or progenitor cells are initially inoculated into said first medium from 1×10⁴ to 1×10⁵ cells/mL. In a specific aspect, said hematopoietic stem or progenitor cells are initially inoculated into said first medium at about 3×10⁴ cells/mL.

In certain aspects, said first population of cells are initially inoculated into said second medium from 5×10⁴ to 5×10⁵ cells/mL. In a specific aspect, said first population of cells is initially inoculated into said second medium at about 1×10⁵ cells/mL.

In certain aspects said second population of cells is initially inoculated into said third medium from 1×10⁵ to 5×10⁶ cells/mL. In a specific aspect, said second population of cells is initially inoculated into said third medium at about 5×10⁵ cells/mL. In a more specific aspect, said second population of cells is initially inoculated into said third medium at about 5×10⁵ cells/mL in a spinner flask. In a specific aspect, said second population of cells is initially inoculated into said third medium at about 3×10⁵ cells/mL. In a more specific aspect, said second population of cells is initially inoculated into said third medium at about 3×10⁵ cells/mL in a static culture.

In a certain embodiment, the three-stage method comprises a first stage (“stage 1”) comprising culturing placenta derived hematopoietic stem cells or progenitor cells, e.g., CD34+ stem cells or progenitor cells, in a first medium for a specified time period, e.g. as described herein, to produce a first population of cells. In certain embodiments, the first medium comprises a stem cell mobilizing agent and thrombopoietin (Tpo). In certain embodiments, the first medium comprises in addition to a stem cell mobilizing agent and Tpo, one or more of LMWH, FIt-3L, SCF, IL-6, IL-7, G-CSF, and GM-CSF. In a specific embodiment, the first medium comprises in addition to a stem cell mobilizing agent and Tpo, each of LMWH, FIt-3L, SCF, IL-6, IL-7, G-CSF, and GM-CSF. In a specific embodiment, the first medium lacks added LMWH. In a specific embodiment, the first medium lacks added desulphated glycosaminoglycans. In a specific embodiment, the first medium lacks LMWH. In a specific embodiment, the first medium lacks desulphated glycosaminoglycans. In a specific embodiment, in addition to a stem cell mobilizing agent and Tpo, each of Flt-3L, SCF, IL-6, IL-7, G-CSF, and GM-CSF. In specific embodiments, the first medium lacks leukemia inhibiting factor (LIF), macrophage inhibitory protein-1 alpha (MIP-la) or both.

In certain embodiments, subsequently, in “stage 2” said cells are cultured in a second medium for a specified time period, e.g. as described herein, to produce a second population of cells. In certain embodiments, the second medium comprises a stem cell mobilizing agent and interleukin-15 (IL-15) and lacks Tpo. In certain embodiments, the second medium comprises, in addition to a stem cell mobilizing agent and IL-15, one or more of LMWH, Flt-3, SCF, IL-6, IL-7, G-CSF, and GM-CSF. In certain embodiments, the second medium comprises, in addition to a stem cell mobilizing agent and IL-15, each of LMWH, Flt-3, SCF, IL-6, IL-7, G-CSF, and GM-CSF. In a specific embodiment, the second medium lacks added LMWH. In a specific embodiment, the second medium lacks added desulphated glycosaminoglycans. In a specific embodiment, the second medium lacks heparin, e.g., LMWH. In a specific embodiment, the second medium lacks desulphated glycosaminoglycans. In certain embodiments, the second medium comprises, in addition to a stem cell mobilizing agent and IL-15, each of Flt-3, SCF, IL-6, IL-7, G-CSF, and GM-CSF. In specific embodiments, the second medium lacks leukemia inhibiting factor (LIF), macrophage inhibitory protein-1 alpha (MIP-la) or both.

Subsequently, in “stage 3” said cells are cultured in a third medium for a specified time period, e.g. as described herein, to produce a third population of cell, e.g., natural killer cells. In certain embodiments, the third medium comprises IL-2 and IL-15, and lacks a stem cell mobilizing agent and LMWH. In certain embodiments, the third medium comprises in addition to IL-2 and IL-15, one or more of SCF, IL-6, IL-7, G-CSF, and GM-CSF. In certain embodiments, the third medium comprises, in addition to IL-2 and IL-15, each of SCF, IL-6, IL-7, G-CSF, and GM-CSF. In specific embodiments, the first medium lacks one, two, or all three of LIF, MIP-la, and FIt3L. In specific embodiments, the third medium lacks added desulphated glycosaminoglycans. In specific embodiments, the third medium lacks desulphated glycosaminoglycans. In specific embodiments, the third medium lacks heparin, e.g., LMWH.

In a specific embodiment, the three-stage method is used to produce placenta derived NK cell populations. In certain embodiments, the three-stage method is conducted in the absence of stromal feeder cell support. In certain embodiments, the three-stage method is conducted in the absence of exogenously added steroids (e.g. cortisone, hydrocortisone, or derivatives thereof).

In certain aspects, said first medium used in the three-stage method comprises a stem cell mobilizing agent and thrombopoietin (Tpo). In certain aspects, the first medium used in the three-stage method comprises, in addition to a stem cell mobilizing agent and Tpo, one or more of Low Molecular Weight Heparin (LMWH), Flt-3 Ligand (FIt-3L), stem cell factor (SCF), IL-6, IL-7, granulocyte colony-stimulating factor (G-CSF), or granulocyte-macrophage-stimulating factor (GM-CSF). In certain aspects, the first medium used in the three-stage method comprises, in addition to a stem cell mobilizing agent and Tpo, each of LMWH, FIt-3L, SCF, IL-6, IL-7, G-CSF, and GM-CSF. In certain aspects, the first medium used in the three-stage method comprises, in addition to a stem cell mobilizing agent and Tpo, each of Flt-3L, SCF, IL-6, IL-7, G-CSF, and GM-CSF. In a specific aspect, the first medium lacks added LMWH. In a specific aspect, the first medium lacks added desulphated glycosaminoglycans. In a specific aspect, the first medium lacks LMWH. In a specific aspect, the first medium lacks desulphated glycosaminoglycans. In certain aspects, said Tpo is present in the first medium at a concentration of from 1 ng/ml to 100 ng/ml, from 1 ng/ml to 50 ng/ml, from 20 ng/mL to 30 ng/mL, or about 25 ng/ml. In other aspects, said Tpo is present in the first medium at a concentration of from 100 ng/ml to 500 ng/ml, from 200 ng/ml to 300 ng/ml, or about 250 ng/ml. In certain aspects, when LMWH is present in the first medium, the LMWH is present at a concentration of from IU/mL to IOU/ml; the Flt-3L is present at a concentration of from 1 ng/ml to 50 ng/ml; the SCF is present at a concentration of from 1 ng/ml to 50 ng/ml; the IL-6 is present at a concentration of from 0.01 ng/ml to 0.1 ng/ml; the IL-7 is present at a concentration of from 1 ng/ml to 50 ng/mL; the G-CSF is present at a concentration of from 0.01 ng/ml to 0.50 ng/ml; and the GM-CSF is present at a concentration of from 0.005 ng/ml to 0.1 ng/ml. In certain aspects, in the first medium, the Flt-3L is present at a concentration of from 1 ng/ml to 50 ng/ML; the SCF is present at a concentration of from 1 ng/ml to 50 ng/ml; the IL-6 is present at a concentration of from 0.01 ng/ml to 0.1 ng/mL; the IL-7 is present at a concentration of from 1 ng/mL to 50 ng/ml; the G-CSF is present at a concentration of from 0.01 ng/ml to 0.50 ng/ml; and the GM-CSF is present at a concentration of from 0.005 ng/ml to 0.1 ng/ml. In certain aspects, when LMWH is present in the first medium, the LMWH is present at a concentration of from 4 U/mL to 5 U/mL; the Flt-3L is present at a concentration of from 20 ng/ml to 30 ng/ml; the SCF is present at a concentration of from 20 ng/ml to 30 ng/ml; the IL-6 is present at a concentration of from 0.04 ng/mL to 0.06 ng/ml; the IL-7 is present at a concentration of from 20 ng/mL to 30 ng/ml; the G-CSF is present at a concentration of from 0.20 ng/ml to 0.30 ng/ML; and the GM-CSF is present at a concentration of from 0.005 ng/ml to 0.5 ng/mL. In certain aspects, in the first medium, the Flt-3L is present at a concentration of from 20 ng/ml to 30 ng/ml; the SCF is present at a concentration of from 20 ng/ml to 30 ng/ml; the IL-6 is present at a concentration of from 0.04 ng/ml to 0.06 ng/ml; the IL-7 is present at a concentration of from 20 ng/ml to 30 ng/ml; the G-CSF is present at a concentration of from 0.20 ng/ml to 0.30 ng/ml; and the GM-CSF is present at a concentration of from 0.005 ng/ml to 0.5 ng/ml. In certain aspects, when LMWH is present in the first medium, the LMWH is present at a concentration of about 4.5 U/mL; the Flt-3L is present at a concentration of about 25 ng/ml; the SCF is present at a concentration of about 27 ng/ml; the IL-6 is present at a concentration of about 0.05 ng/ml; the IL-7 is present at a concentration of about 25 ng/ml; the G-CSF is present at a concentration of about 0.25 ng/ML; and the GM-CSF is present at a concentration of about 0.01 ng/ml. In certain aspects, in the first medium, the Flt-3L is present at a concentration of about 25 ng/ml; the SCF is present at a concentration of about 27 ng/ml; the IL-6 is present at a concentration of about 0.05 ng/ml; the IL-7 is present at a concentration of about 25 ng/ml; the G-CSF is present at a concentration of about 0.25 ng/ml; and the GM-CSF is present at a concentration of about 0.01 ng/ml. In certain embodiments, said first medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the first medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g. 2:1 ratio, or high glucose or low glucose DMEM), Advanced DMEM (Gibco), EL08-1D2, Myelocult™ H5100, IMDM, and/or RPMI-1640. In certain embodiments, said first medium is not GBGM®. In specific embodiments of any of the above embodiments, the first medium lacks LIF, MIP-la, or both.

In certain aspects, said second medium used in the three-stage method comprises a stem cell mobilizing agent and interleukin-15 (IL-15), and lacks Tpo. In certain aspects, the second medium used in the three-stage method comprises, in addition to a stem cell mobilizing agent and IL-15, one or more of LMWH, FIt-3, SCF, IL-6, IL-7, G-CSF, and GM-CSF. In certain aspects, the second medium used in the three-stage method comprises, in addition to a stem cell mobilizing agent and IL-15, each of LMWH, Flt-3, SCF, IL-6, IL-7, G-CSF, and GM-CSF. In certain aspects, the second medium used in the three-stage method comprises, in addition to a stem cell mobilizing agent and IL-15, each of Flt-3, SCF, IL-6, IL-7, G-CSF, and GM-CSF. In a specific aspect, the second medium lacks added LMWH. In a specific aspect, the second medium lacks added desulphated glycosaminoglycans. In a specific aspect, the second medium lacks LMWH. In a specific aspect, the second medium lacks desulphated glycosaminoglycans. In certain aspects, said IL-15 is present in said second medium at a concentration of from 1 ng/ml to 50 ng/ml, from 10 ng/ml to 30 ng/ml, or about 20 ng/ml. In certain aspects, when LMWH is present in said second medium, the LMWH is present at a concentration of from IU/mL to IOU/ml; the Flt-3L is present at a concentration of from 1 ng/mL to 50 ng/ml; the SCF is present at a concentration of from 1 ng/ml to 50 ng/ml; the IL-6 is present at a concentration of from 0.01 ng/ml to 0.1 ng/ml; the IL-7 is present at a concentration of from 1 ng/ml to 50 ng/ml; the G-CSF is present at a concentration of from 0.01 ng/ml to 0.50 ng/ml; and the GM-CSF is present at a concentration of from 0.005 ng/ml to 0.1 ng/mL. In certain aspects, in said second medium, the Flt-3L is present at a concentration of from 1 ng/ml to 50 ng/ml; the SCF is present at a concentration of from 1 ng/ml to 50 ng/ml; the IL-6 is present at a concentration of from 0.01 ng/ml to 0.1 ng/ml; the IL-7 is present at a concentration of from 1 ng/ml to 50 ng/ml; the G-CSF is present at a concentration of from 0.01 ng/ml to 0.50 ng/ml; and the GM-CSF is present at a concentration of from 0.005 ng/ml to 0.1 ng/ml. In certain aspects, when LMWH is present in the second medium, the LMWH is present in the second medium at a concentration of from 4 U/mL to 5 U/ml; the Flt-3L is present at a concentration of from 20 ng/mL to 30 ng/ml; the SCF is present at a concentration of from 20 ng/ml to 30 ng/ml; the IL-6 is present at a concentration of from 0.04 ng/mL to 0.06 ng/ml; the IL-7 is present at a concentration of from 20 ng/ml to 30 ng/ml; the G-CSF is present at a concentration of from 0.20 ng/ml to 0.30 ng/mL; and the GM-CSF is present at a concentration of from 0.005 ng/ml to 0.5 ng/mL. In certain aspects, in the second medium, the Flt-3L is present at a concentration of from 20 ng/ml to 30 ng/mL; the SCF is present at a concentration of from 20 ng/ml to 30 ng/ml; the IL-6 is present at a concentration of from 0.04 ng/ml to 0.06 ng/ML; the IL-7 is present at a concentration of from 20 ng/ml to 30 ng/ml; the G-CSF is present at a concentration of from 0.20 ng/ml to 0.30 ng/ml; and the GM-CSF is present at a concentration of from 0.005 ng/mL to 0.5 ng/ml. In certain aspects, when LMWH is present in the second medium, the LMWH is present in the second medium at a concentration of from 4 U/mL to 5 U/mL; the Flt-3L is present at a concentration of from 20 ng/mL to 30 ng/ml; the SCF is present at a concentration of from 20 ng/ml to 30 ng/ml; the IL-6 is present at a concentration of from 0.04 ng/ml to 0.06 ng/ml; the IL-7 is present at a concentration of from 20 ng/ml to 30 ng/ml; the G-CSF is present at a concentration of from 0.20 ng/ml to 0.30 ng/ml; and the GM-CSF is present at a concentration of from 0.005 ng/ml to 0.5 ng/ml. In certain aspects, in the second medium, the Flt-3L is present at a concentration of from 20 ng/ml to 30 ng/ml; the SCF is present at a concentration of from 20 ng/ml to 30 ng/mL; the IL-6 is present at a concentration of from 0.04 ng/mL to 0.06 ng/ml; the IL-7 is present at a concentration of from 20 ng/ml to 30 ng/ml; the G-CSF is present at a concentration of from 0.20 ng/ml to 0.30 ng/ml; and the GM-CSF is present at a concentration of from 0.005 ng/mL to 0.5 ng/ml. In certain aspects, when LMWH is present in the second medium, the LMWH is present in the second medium at a concentration of about 4.5 U/mL; the Flt-3L is present at a concentration of about 25 ng/ml; the SCF is present at a concentration of about 27 ng/ml; the IL-6 is present at a concentration of about 0.05 ng/ml; the IL-7 is present at a concentration of about 25 ng/ml; the G-CSF is present at a concentration of about 0.25 ng/ml; and the GM-CSF is present at a concentration of about 0.01 ng/ml. In certain aspects, in the second medium, the Flt-3L is present at a concentration of about 25 ng/ml; the SCF is present at a concentration of about 27 ng/ml; the IL-6 is present at a concentration of about 0.05 ng/ml; the IL-7 is present at a concentration of about 25 ng/ml; the G-CSF is present at a concentration of about 0.25 ng/ml; and the GM-CSF is present at a concentration of about 0.01 ng/ml. In certain embodiments, said second medium additionally comprises one or more of the following: antibiotics such as gentamycin; antioxidants such as transferrin, insulin, and/or betamercaptoethanol; sodium selenite; ascorbic acid; ethanolamine; and glutathione. In certain embodiments, the medium that provides the base for the second medium is a cell/tissue culture medium known to those of skill in the art, e.g., a commercially available cell/tissue culture medium such as SCGM™, STEMMACS™, GBGM®, AIM-VR, X-VIVO™ 10, X-VIVO™ 15, OPTMIZER, STEMSPAN® H3000, CELLGRO COMPLETE™, DMEM:Ham's F12 (“F12”) (e.g. 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-VR, X-VIVO™ 10, X-VIVO™ 15, OPTMIZER, STEMSPAN® H3000, CELLGRO COMPLETE™, DMEM:Ham's F12 (“F12”) (e.g. 2:1 ratio, or high glucose or low glucose DMEM), Advanced DMEM (Gibco), EL08-1D2, Myelocult™ H5100, IMDM, and/or RPMI-1640. In certain embodiments, said second medium is not GBGMR. In specific embodiments of any of the above embodiments, the first medium lacks LIF, MIP-la, or both.

The third medium used in the three-stage method to produce placental derived NK cells having applications in the instant disclosure comprises IL-2 and IL-15, and lacks a stem cell mobilizing agent and LMWH. In certain aspects, said third medium used in the three-stage method comprises IL-2 and IL-15, and lacks LMWH. In certain aspects, said third medium used in the three-stage method comprises IL-2 and IL-15, and lacks SCF and LMWH. In certain aspects, said third medium used in the three-stage method comprises IL-2 and IL-15, and lacks SCF, a stem cell mobilizing agent and LMWH. In certain aspects, said third medium used in the three-stage method comprises a stem cell mobilizing agent, IL-2 and IL-15, and lacks LMWH. In certain aspects, said third medium used in the three-stage method comprises SCF, IL-2 and IL-15, and lacks LMWH. In certain aspects, said third medium used in the three-stage method comprises a stem cell mobilizing agent, SCF, IL-2 and IL-15, and lacks LMWH. In certain aspects, said third medium used in the three-stage method comprises IL-2 and IL-15, and lacks a stem cell mobilizing agent and LMWH. In certain aspects, the third medium used in the three-stage method comprises, in addition to IL-2 and IL-15, one or more of SCF, IL-6, IL-7, G-CSF, or GM-CSF. In certain aspects, the third medium used in the three-stage method comprises, in addition to IL-2 and IL-15, each of SCF, IL-6, IL-7, G-CSF, and GM-CSF. In certain aspects, said IL-2 is present in said third medium at a concentration of from 10 U/mL to 10,000 U/mL and said IL-15 is present in said third medium at a concentration of from 1 ng/mL to 50 ng/ml. In certain aspects, said IL-2 is present in said third medium at a concentration of from 100 U/ml to 10,000 U/ml and said IL-15 is present in said third medium at a concentration of from 1 ng/ml to 50 ng/ml. In certain aspects, said IL-2 is present in said third medium at a concentration of from 300 U/mL to 3,000 U/mL and said IL-15 is present in said third medium at a concentration of from 10 ng/mL to 30 ng/mL. In certain aspects, said IL-2 is present in said third medium at a concentration of about 1,000 U/mL and said IL-15 is present in said third medium at a concentration of about 20 ng/ml. In certain aspects, in said third medium, the SCF is present at a concentration of from 1 ng/mL to 50 ng/ml; the IL-6 is present at a concentration of from 0.01 ng/ml to 0.1 ng/ml; the IL-7 is present at a concentration of from 1 ng/ml to 50 ng/ml; the G-CSF is present at a concentration of from 0.01 ng/ml to 0.50 ng/ml; and the GM-CSF is present at a concentration of from 0.005 ng/ml to 0.1 ng/ml. In certain aspects, in said third medium, the SCF is present at a concentration of from 20 ng/ml to 30 ng/ml; the IL-6 is present at a concentration of from 0.04 ng/ml to 0.06 ng/ml; the IL-7 is present at a concentration of from 20 ng/ml to 30 ng/ML; the G-CSF is present at a concentration of from 0.20 ng/ml to 0.30 ng/ml; and the GM-CSF is present at a concentration of from 0.005 ng/ml to 0.5 ng/ml. In certain aspects, in said third medium, the SCF is present at a concentration of about 22 ng/ML; the IL-6 is present at a concentration of about 0.05 ng/ml; the IL-7 is present at a concentration of about 20 ng/ml; the G-CSF is present at a concentration of about 0.25 ng/ml; and the GM-CSF is present at a concentration of about 0.01 ng/ml. In certain aspects, the third medium comprises 100 ng/ml IL-7, 1000 ng/ml IL-2, 20 ng/ml IL-15, and 10 stem cell mobilizing agent and lacks SCF. In certain aspects, the third medium comprises 20 ng/ML IL-7, 1000 ng/ml IL-2, 20 ng/ml IL-15, and stem cell mobilizing agent and lacks SCF. In certain aspects, the third medium comprises 20 ng/ml IL-7, 20 ng/ml IL-15, and stem cell mobilizing agent and lacks SCF. In certain aspects, the third medium comprises 100 ng/ml IL-7, 22 ng/mL SCF, 1000 ng/ml IL-2, and 20 ng/ml IL-15 and lacks stem cell mobilizing agent. In certain aspects, the third medium comprises 22 ng/ml SCF, 1000 ng/ml IL-2, and 20 ng/ml IL-15 and lacks stem cell mobilizing agent. In certain aspects, the third medium comprises 20 ng/ml IL-7, 22 ng/ml SCF, 1000 ng/ml IL-2, and 20 ng/ml IL-15 and lacks stem cell mobilizing agent. In certain aspects, the third medium comprises 20 ng/ml IL-7, 22 ng/ml SCF, and 1000 ng/ml IL-2 and lacks stem cell mobilizing agent. In specific embodiments of any of the above embodiments, the first medium lacks one, two, or all three of LIF, MIP-la, Flt-3L.

In certain embodiments, the third medium may further comprise one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the third medium is a cell/tissue culture medium known to those of skill in the art, e.g. a commercially available cell/tissue culture medium such as SCGM™, STEMMACS™, GBGM®, AIM-VR, X-VIVO™ 10, X-VIVO™™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g. 2:1 ratio, or high glucose or low glucose DMEM), Advanced DMEM (Gibco), EL08-1D2, Myelocult™ H5100, IMDM, and/or RPMI-1640. In certain embodiments, said third medium is not GBGMR.

Generally, the particularly recited medium components do not refer to possible constituents in an undefined component of said medium. For example, said Tpo, IL-2, and IL-15 are not comprised within an undefined component of the first medium, second medium or third medium, e.g. said Tpo, IL-2, and IL-15 are not comprised within serum. Further, said LMWH, FIt-3, SCF, IL-6, IL-7, G-CSF, and/or GM-CSF are not comprised within an undefined component of the first medium, second medium or third medium, e.g. said LMWH, FIt-3, SCF, IL-6, IL-7, G-CSF, and/or GM-CSF are not comprised within serum.

In certain aspects, said first medium, second medium or third medium comprises human serum-AB. In certain aspects, any of said first medium, second medium or third medium comprises 1% to 20% human serum-AB, 5% to 15% human serum-AB, or about 2, 5, or 10% human serum-AB. In certain embodiments, in the three-stage methods described herein, said hematopoietic stem or progenitor cells are cultured in said first medium for 1, 2, 3, 4, 5, 6, 7, 8, 9, 10, 11, 12, 13, 14, 15, 16, 17, 18, 19, or 20 days. In certain embodiments, in the three-stage methods described herein, cells are cultured in said second medium for 1, 2, 3, 4, 5, 6, 7, 8, 9, 10, 11, 12, 13, 14, 15, 16, 17, 18, 19, or 20 days. In certain embodiments, in the three-stage methods described herein, cells are cultured in said third medium for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or for more than 30 days.

In a specific embodiment, in the three-stage methods described herein, the placental derived hematopoietic stem or progenitor cells are cultured in said first medium for 7-13 days to produce a first population of cells, before said culturing in said second medium; said first population of cells are cultured in said second medium for 2-6 days to produce a second population of cells before said culturing in said third medium; and said second population of cells are cultured in said third medium for 10-30 days, i.e., the cells are cultured a total of 19-49 days.

In a specific embodiment, in the three-stage methods described herein, in the three-stage methods described herein, said hematopoietic stem or progenitor cells are cultured in said first medium for 8-12 days to produce a first population of cells, before said culturing in said second medium; said first population of cells are cultured in said second medium for 3-5 days to produce a second population of cells before said culturing in said third medium; and said second population of cells are cultured in said third medium for 15-25 days, i.e., the cells are cultured a total of 26-42 days.

In a specific embodiment, in the three-stage methods described herein, said hematopoietic stem or progenitor cells are cultured in said first medium for about 10 days to produce a first population of cells, before said culturing in said second medium; said first population of cells are cultured in said second medium for about 4 days to produce a second population of cells before said culturing in said third medium; and said second population of cells are cultured in said third medium for about 21 days, i.e., the cells are cultured a total of about 35 days.

In certain aspects, the three-stage method disclosed herein produces at least 5000-fold more placenta derived NK cells having applications in the instant disclosure as compared to the number of hematopoietic stem cells initially inoculated into said first medium. In certain aspects, said three-stage method produces at least 10,000-fold more natural killer cells as compared to the number of hematopoietic stem cells initially inoculated into said first medium. In certain aspects, said three-stage method produces at least 50,000-fold more natural killer cells as compared to the number of hematopoietic stem cells initially inoculated into said first medium. In certain aspects, said three-stage method produces at least 75,000-fold more natural killer cells as compared to the number of hematopoietic stem cells initially inoculated into said first medium. In certain aspects, the viability of said natural killer cells is determined by 7-aminoactinomycin D (7AAD) staining. In certain aspects, the viability of said natural killer cells is determined by annexin-V staining. In specific aspects, the viability of said natural killer cells is determined by both 7-AAD staining and annexin-V staining. In certain aspects, the viability of said natural killer cells is determined by trypan blue staining.

In certain aspects, the three-stage method produces natural killer cells that comprise at least 20% CD56+CD3− natural killer cells. In certain aspects, the three-stage method produces natural killer cells that comprise at least 40% CD56+CD3− natural killer cells. In certain aspects, the three-stage method produces natural killer cells that comprise at least 60% CD56+CD3− natural killer cells. In certain aspects, the three-stage method produces natural killer cells that comprise at least 70% CD56+CD3− natural killer cells. In certain aspects, the three-stage method produces natural killer cells that comprise at least 80% CD56+CD3− natural killer cells.

In certain aspects, the three-stage method disclosed herein produces natural killer cells that comprise at least 20% CD56+CD3− CDlla+ natural killer cells. In certain aspects, the three-stage method disclosed herein produces natural killer cells that comprise at least 40% CD56+CD3− CD1 la+ natural killer cells. In certain aspects, the three-stage method disclosed herein produces natural killer cells that comprise at least 60% CD56+CD3− CDlla+ natural killer cells. In certain aspects, the three-stage method disclosed herein produces natural killer cells that comprise at least 80% CD56+CD3− CDlla+ natural killer cells.

In certain aspects, the three-stage method disclosed herein produces ILC3 cells that comprise at least 20% CD56+CD3− CD1 la-ILC3 cells. In certain aspects, the three-stage method disclosed herein produces ILC3 cells that comprise at least 40% CD56+CD3− CDlla-ILC3 cells. In certain aspects, the three-stage method disclosed herein produces ILC3 cells that comprise at least 60% CD56+CD3− CDlla-ILC3 cells. In certain aspects, the three-stage method disclosed herein produces natural killer cells that comprise at least 80% CD56+CD3− CDlla-ILC3 cells.

In certain aspects, the three-stage method produces natural killer cells that exhibit at least 20% cytotoxicity against K562 cells when said natural killer cells and said K562 cells are co-cultured in vitro or ex vivo at a ratio of 10:1. In certain aspects, the three-stage method produces natural killer cells that exhibit at least 35% cytotoxicity against the K562 cells when said natural killer cells and said K562 cells are co-cultured in vitro or ex vivo at a ratio of 10:1. In certain aspects, the three-stage method produces natural killer cells that exhibit at least 45% cytotoxicity against the K562 cells when said natural killer cells and said K562 cells are co-cultured in vitro or ex vivo at a ratio of 10:1. In certain aspects, the three-stage method produces natural killer cells that exhibit at least 60% cytotoxicity against the K562 cells when said natural killer cells and said K562 cells are co-cultured in vitro or ex vivo at a ratio of 10:1. In certain aspects, the three-stage method produces natural killer cells that exhibit at least 75% cytotoxicity against the K562 cells when said natural killer cells and said K562 cells are co-cultured in vitro or ex vivo at a ratio of 10:1.

In certain aspects, provided herein are populations of cells comprising natural killer cells, i.e., natural killers cells produced by a three-stage method described herein. Accordingly, provided herein is an isolated natural killer cell population produced by a three-stage method described herein. In a specific embodiment, said natural killer cell population comprises at least 20% CD56+CD3− natural killer cells. In a specific embodiment, said natural killer cell population comprises at least 40% CD56+CD3− natural killer cells. In a specific embodiment, said natural killer cell population comprises at least 60% CD56+CD3− natural killer cells. In a specific embodiment, said natural killer cell population comprises at least 80% CD56+CD3− natural killer cells. In a specific embodiment, said natural killer cell population comprises at least 60% CD16-cells. In a specific embodiment, said natural killer cell population comprises at least 80% CD16-cells. In a specific embodiment, said natural killer cell population comprises at least 20% CD94+ cells. In a specific embodiment, said natural killer cell population comprises at least 40% CD94+ cells.

In certain aspects, provided herein is a population of natural killer cells that is CD56+CD3− CD117+CD1 la+, wherein said natural killer cells express perforin and/or EOMES, and do not express one or more of RORyt, aryl hydrocarbon receptor (AHR), and IL1R1. In certain aspects, said natural killer cells express perforin and EOMES, and do not express any of RORyt, aryl hydrocarbon receptor, or IL1R1. In certain aspects, said natural killer cells additionally express T-bet, GZMB, NKp46, NKp30, and NKG2D. In certain aspects, said natural killer cells express CD94. In certain aspects, said natural killer cells do not express CD94.

Thus, a method of producing a cell population comprising placeneta derived natural killer cells comprises: (a) culturing placenta derived hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) separating CD1 la+ cells and CD1 la-cells from the third population of cells; and (e) combining the CDlla+ cells with the CDlla-cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50 to produce a fourth population of cells. In certain embodiments, said first medium and/or said second medium lack leukemia inhibiting factor (LIF) and/or macrophage inflammatory protein-1 alpha (MIP-la). In certain embodiments, said third medium lacks LIF, MIP-la, and FMS-like tyrosine kinase-3 ligand (Flt-3L). In specific embodiments, said first medium and said second medium lack LIF and MIP-la, and said third medium lacks LIF, MIP-la, and Fit3L. In certain embodiments, none of the first medium, second medium or third medium comprises heparin, e.g., low-molecular weight heparin. In certain aspects, in the fourth population of cells, the CD1 la+ cells and CDlla-cells are combined in a ratio of 50:1. In certain aspects, in the fourth population of cells, the CDlla+ cells and CDlla-cells are combined in a ratio of 20:1. In certain aspects, in the fourth population of cells, the CD1 la+ cells and CD1 la-cells are combined in a ratio of 10:1. In certain aspects, in the fourth population of cells, the CD1 la+ cells and CD1 la-cells are combined in a ratio of 5:1. In certain aspects, in the fourth population of cells, the CD 11 a+ cells and CD I la-cells are combined in a ratio of 1:1. In certain aspects, in the fourth population of cells, the CD1 la+ cells and CD1 la-cells are combined in a ratio of 1:5. In certain aspects, in the fourth population of cells, the CD1 la+ cells and CD1 la-cells are combined in a ratio of 1:10. In certain aspects, in the fourth population of cells, the CD11 a+ cells and CD1 la-cells are combined in a ratio of 1:20. In certain aspects, in the fourth population of cells, the CDlla+ cells and CDlla-cells are combined in a ratio of 1:50.

### Isolation of NK Cells

Methods of isolating natural killer cells are known in the art and can be used to isolate the natural killer cells, e.g. NK cells produced using the three-stage method, described herein. For example, NK cells can be isolated or enriched, for example, by staining cells, in one embodiment, with antibodies to CD56 and CD3, and selecting for CD56 CD3 cells. In certain embodiments, the NK cells are enriched for CD56 CD3 cells in comparison with total cells produced using the three-stage method, described herein. NK cells, e.g. cells produced using the three-stage method, described herein, can be isolated using a commercially available kit, for example, the NK Cell Isolation Kit (Miltenyi Biotec). NK cells, e.g. cells produced using the three-stage method, described herein, can also be isolated or enriched by removal of cells other than NK cells in a population of cells that comprise the NK cells, e.g., cells produced using the three-stage method, described herein. For example, NK cells, e.g., cells produced using the three-stage method, described herein, may be isolated or enriched by depletion of cells displaying non-NK cell markers using, e.g., antibodies to one or more of CD3, CD4, CD14, CD19, CD20, CD36, CD66b, CD123, HLA DR and/or CD235a (glycophorin A). Negative isolation can be carried out using a commercially available kit, e.g., the NK Cell Negative Isolation Kit (Dynal Biotech). Cells isolated by these methods may be additionally sorted, e.g., to separate CDlla+ and CDI la-cells, and/or CD117+ and CD117-cells, and/or CD16+ and CD 16 cells, and/or CD94+ and CD94″. In certain embodiments, cells, e.g. cells produced by the three-step methods described herein, are sorted to separate CD1 la+ and CD1 la-cells. In specific embodiments, CD1 la+ cells are isolated. In certain embodiments, the cells are enriched for CD1 la+ cells in comparison with total cells produced using the three-stage method, described herein. In specific embodiments, CD1 la-cells are isolated. In certain embodiments, the cells are enriched for CD1 la-cells in comparison with total cells produced using the three-stage method, described herein. In certain embodiments, cells are sorted to separate CD117+ and CD117-cells. In specific embodiments, CD117+ cells are isolated. In certain embodiments, the cells are enriched for CD117+ cells in comparison with total cells produced using the three-stage method, described herein. In specific embodiments, CD117-cells are isolated. In certain embodiments, the cells are enriched for CD117-cells in comparison with total cells produced using the three-stage method, described herein. In certain embodiments, cells are sorted to separate CD16+ and CD 16 cells. In specific embodiments, CD16+ cells are isolated. In certain embodiments, the cells are enriched for CD16+ cells in comparison with total cells produced using the three-stage method, described herein. In specific embodiments, CD 16 cells are isolated. In certain embodiments, the cells are enriched for CD 16-cells in comparison with total cells produced using the three-stage method, described herein. In certain embodiments, cells are sorted to separate CD94+ and CD94 cells. In specific embodiments, CD94+ cells are isolated. In certain embodiments, the cells are enriched for CD94+ cells in comparison with total cells produced using the three-stage method, described herein. In specific embodiments, CD94 cells are isolated. In certain embodiments, the cells are enriched for CD94-cells in comparison with total cells produced using the three-stage method, described herein. In certain embodiments, isolation is performed using magnetic separation. In certain embodiments, isolation is performed using flow cytometry.

In one embodiment, ILC3 cells are isolated or enriched by selecting for CD56 CD3 CD I la cells. In certain embodiments, the ILC3 cells are enriched for CD56 CD3 CD I la cells in comparison with total cells produced using the three-stage method, described herein. In one embodiment, ILC3 cells are isolated or enriched by selecting for CD56 CD3 CD I I a CD 117+ cells. In certain embodiments, the ILC3 cells are enriched for CD56 CD3 CD 1 1 a CD I 17+ cells in comparison with total cells produced using the three-stage method, described herein. In one embodiment, ILC3 cells are isolated or enriched by selecting for CD56 CD3 CD I I a CD 1 17 CDILI R 1 1 cells. In certain embodiments, the ILC3 cells are enriched for CD56 CD3 CD I la CD I I 7 CDIL I R I 1 cells in comparison with total cells produced using the three-stage method, described herein.

In one embodiment, NK cells are isolated or enriched by selecting for CD56 CD3 CD94 CD I la+ cells. In certain embodiments, the NK cells are enriched for CD56 CD3 CD94 CD I la+ cells in comparison with total cells produced using the three-stage method, described herein. In one embodiment, NK cells are isolated or enriched by selecting for CD56 CD3 CD94 CD 1 1 a′CD 1 17 cells. In certain embodiments, the NK cells are enriched for CD56 CD3 CD94 CD I I a′CD 1 17 cells in comparison with total cells produced using the three-stage method, described herein.

Cell separation can be accomplished by, e.g., flow cytometry, fluorescence-activated cell sorting (FACS), or, in one embodiment, magnetic cell sorting using microbeads conjugated with specific antibodies. The cells may be isolated, e.g., using a magnetic activated cell sorting (MACS) technique, a method for separating particles based on their ability to bind magnetic beads (e.g., about 0.5-100 μm diameter) that comprise one or more specific antibodies, e.g., anti-CD56 antibodies. Magnetic cell separation can be performed and automated using, e.g., an AUTOMACS™ Separator (Miltenyi). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

### Placental Perfusate

Placenta derived NK cells or a population thereof produced according to the three-stage method described herein may be produced from hematopoietic cells, e.g., hematopoietic stem or progenitors from any placental source, e.g., placental tissue, placental perfusate, umbilical cord blood, placental blood. In certain embodiments, the hematopoietic stem cells are combined hematopoietic stem cells from placental perfusate and from cord blood from the same placenta used to generate the placental perfusate. Placental perfusate comprising placental perfusate cells that can be obtained, for example, by the methods disclosed in U.S. Pat. Nos. 7,045,148 and 7,468,276 and U.S. Patent Application Publication No. 2009/0104164, the disclosures of which are hereby incorporated in their entireties.

### Cell Collection Composition

The placental perfusate and perfusate cells, from which hematopoietic stem or progenitors may be isolated, produced according to the three-stage method provided herein and having applications in the instant disclosure, can be collected by perfusion of a mammalian, e.g., human postpartum placenta using a placental cell collection composition. Perfusate can be collected from the placenta by perfusion of the placenta with any physiologically-acceptable solution, e.g., a saline solution, culture medium, or a more complex cell collection composition. A cell collection composition suitable for perfusing a placenta, and for the collection and preservation of perfusate cells is described in detail in related U.S. Application Publication No. 2007/0190042, which is incorporated herein by reference in its entirety.

The cell collection composition can comprise any physiologically-acceptable solution suitable for the collection and/or culture of stem cells, for example, a saline solution (e.g., phosphate-buffered saline, Kreb's solution, modified Kreb's solution, Eagle's solution, 0.9% NaCl, etc.), a culture medium (e.g., DMEM, H.DMEM, etc.), and the like.

The cell collection composition can comprise one or more components that tend to preserve placental cells, that is, prevent the placental cells from dying, or delay the death of the placental cells, reduce the number of placental cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.) a necrosis inhibitor (e.g., 2-(IH-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, a hyaluronidase, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram (+) and/or Gram (−) bacteria, e.g., Pseudomonas aeruginosa, Staphylococcus aureus, and the like.

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/1, or about 40 g/l to about 60 g/1); an antioxidant (e.g., butylated hydroxyanisole, butylated hydroxytoluene, glutathione, vitamin C or vitamin E present at about 25 pM to about 100 pM); a reducing agent (e.g., N-acetylcysteine present at about 0.1 mM to about 5 mM); an agent that prevents calcium entry into cells (e.g., verapamil present at about 2 pM to about 25 pM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/1 to about 100,000 units/1); or an amiloride containing compound (e.g. amiloride, ethyl isopropyl amiloride, hexamethylene amiloride, dimethyl amiloride or isobutyl amiloride present at about 1.0 pM to about 5 pM).

### Collection and Handling of Placenta

Generally, a human placenta is recovered shortly after its expulsion after birth. In one embodiment, the placenta is recovered from a patient after informed consent and after a complete medical history of the patient is taken and is associated with the placenta. In one embodiment, the medical history continues after delivery.

Prior to recovery of perfusate, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank Inc., Cedar Knolls, N.J., ViaCord, Cord Blood Registry and CryoCell. In one embodiment, the placenta is gravity drained without further manipulation so as to minimize tissue disruption during cord blood recovery.

Typically, a placenta is transported from the delivery or birthing room to another location, e.g., a laboratory, for recovery of cord blood and collection of perfusate. The placenta can be transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in U.S. Pat. No. 7,147,626. In one embodiment, the placenta is delivered to the laboratory four to twenty-four hours following delivery. In certain embodiments, the proximal umbilical cord is clamped, for example within 4-5 cm (centimeter) of the insertion into the placental disc prior to cord blood recovery. In other embodiments, the proximal umbilical cord is clamped after cord blood recovery but prior to further processing of the placenta.

The placenta, prior to collection of the perfusate, can be stored under sterile conditions and at either room temperature or at a temperature of 5 to 25° C. (centigrade). The placenta may be stored for a period of longer than forty eight hours, or for a period of four to twenty-four hours prior to perfusing the placenta to remove any residual cord blood. The placenta can be stored in an anticoagulant solution at a temperature of 5° C. to 25° C. (centigrade). Suitable anticoagulant solutions are well known in the art. For example, a solution of heparin or warfarin sodium can be used. In one embodiment, the anticoagulant solution comprises a solution of heparin (e.g., 1% w/w in 1:1000 solution). In some embodiments, the exsanguinated placenta is stored for no more than 36 hours before placental perfusate is collected.

### Placental Perfusion

Methods of perfusing mammalian placentae and obtaining placental perfusate are disclosed, e.g., in Hariri, U.S. Pat. Nos. 7,045,148 and 7,255,879, and in U.S. Application Publication Nos. 2009/0104164, 2007/0190042 and 20070275362, issued as U.S. Pat. No. 8,057,788, the disclosures of which are hereby incorporated by reference herein in their entireties.

Perfusate can be obtained by passage of perfusion solution, e.g., saline solution, culture medium or cell collection compositions described above, through the placental vasculature. In one embodiment, a mammalian placenta is perfused by passage of perfusion solution through either or both of the umbilical artery and umbilical vein. The flow of perfusion solution through the placenta may be accomplished using, e.g., gravity flow into the placenta. For example, the perfusion solution is forced through the placenta using a pump, e.g., a peristaltic pump. The umbilical vein can be, e.g., cannulated with a cannula, e.g., a TEFLON or plastic cannula, that is connected to a sterile connection apparatus, such as sterile tubing. The sterile connection apparatus is connected to a perfusion manifold.

In preparation for perfusion, the placenta can be oriented in such a manner that the umbilical artery and umbilical vein are located at the highest point of the placenta. The placenta can be perfused by passage of a perfusion solution through the placental vasculature, or through the placental vasculature and surrounding tissue. In one embodiment, the umbilical artery and the umbilical vein are connected simultaneously to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. In another embodiment, the perfusion solution is passed through the umbilical veins and collected from the umbilical artery, or is passed through the umbilical artery and collected from the umbilical veins, that is, is passed through only the placental vasculature (fetal tissue).

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. Placental cells collected by this method, which can be referred to as a “closed circuit” method, are typically almost exclusively fetal.

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 ml of perfusion solution. Cells in the perfusate are then collected, e.g., by centrifugation.

In one embodiment, the proximal umbilical cord is clamped during perfusion, and, more specifically, can be clamped within 4-5 cm (centimeter) of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 mL of perfusion fluid is adequate to initially flush blood from the placenta, but more or less perfusion fluid may be used depending on the observed results.

In certain embodiments, cord blood is removed from the placenta prior to perfusion (e.g., by gravity drainage), but the placenta is not flushed (e.g., perfused) with solution to remove residual blood. In certain embodiments, cord blood is removed from the placenta prior to perfusion (e.g., by gravity drainage), and the placenta is flushed (e.g. perfused) with solution to remove residual blood.

The volume of perfusion liquid used to perfuse the placenta may vary depending upon the number of placental cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 ml to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with a cell collection composition, or a standard perfusion solution (e.g., a normal saline solution such as phosphate buffered saline (“PBS”) with or without an anticoagulant (e.g. heparin, warfarin sodium, coumarin, bishydroxy coumarin), and/or with or without an antimicrobial agent (e.g., P-mercaptoethanol (0.1 mM); antibiotics such as streptomycin (e.g. at 40-100 μg/ml), penicillin (e.g. at 40 U/ml), amphotericin B (e.g. at 0.5 pg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In one embodiment, perfusion of the placenta and collection of perfusion solution, e.g., placental cell collection composition, is repeated until the number of recovered nucleated cells falls below 100 cells/ml. The perfusates at different time points can be further processed individually to recover time-dependent populations of cells, e.g., total nucleated cells. Perfusates from different time points can also be pooled.

### Placental Perfusate and Placental Perfusate Cells

Typically, placental perfusate from a single placental perfusion comprises about 100 million to about 500 million nucleated cells, including hematopoietic cells from which NK cells and/or ILC3 cells, e.g., NK cells and/or ILC3 cells produced according to the three-stage method described herein, may be produced by the method disclosed herein. In certain embodiments, the placental perfusate or perfusate cells comprise CD34+ cells, e.g., hematopoietic stem or progenitor cells. Such cells can, in a more specific embodiment, comprise CD341 CD45 stem or progenitor cells, CD34+CD45+ stem or progenitor cells, or the like. In certain embodiments, the perfusate or perfusate cells are cryopreserved prior to isolation of hematopoietic cells therefrom. In certain other embodiments, the placental perfusate comprises, or the perfusate cells comprise, only fetal cells, or a combination of fetal cells and maternal cells.

### NK Cells

### NK Cells Produced by Three-Stage Method

In another embodiment, provided herein is an isolated placental derived NK cell or population thereof having applications in the instant disclosure, wherein said NK cells are produced according to the three-stage method described above. In one embodiment, provided herein is an isolated NK cell population produced by a three-stage method described herein, wherein said NK cell population comprises a greater percentage of CD3−CD56+ cells than an NK progenitor cell population produced by a three-stage method described herein, e.g., an NK progenitor cell population produced by the same three-stage method with the exception that the third culture step used to produce the NK progenitor cell population was of shorter duration than the third culture step used to produce the NK cell population. In a specific embodiment, said NK cell population comprises about 70% or more, in some embodiments, 75%, 80%, 85%, 90%, 95%, 98%, or 99% CD3− CD56+ cells. In another specific embodiment, said NK cell population comprises no less than 80%, 85%, 90%, 95%, 98%, or 99% CD3− CD56+ cells. In another specific embodiment, said NK cell population comprises between 70%-75%, 75%-80%, 80%-85%, 85%-90%, 90%-95%, or 95%-99% CD3− CD56+ cells.

In certain embodiments, said CD3 CD561 cells in said NK cell population comprises CD3 CD561 cells that are additionally NKp46+. In certain embodiments, said CD3 CD561 cells in said NK cell population comprises CD3 CD561 cells that are additionally CD16−. In certain embodiments, said CD3 CD561 cells in said NK cell population comprises CD3 CD561 cells that are additionally CD16+. In certain embodiments, said CD3 CD561 cells in said NK cell population comprises CD3 CD561 cells that are additionally CD94−. In certain embodiments, said CD3 CD561 cells in said NK cell population comprises CD3 CD561 cells that are additionally CD94+. In certain embodiments, said CD3 CD561 cells in said NK cell population comprises CD3 CD561 cells that are additionally CD1 la+. In certain embodiments, said CD3 CD561 cells in said NK cell population comprises CD3 CD561 cells that are additionally NKp30+. In certain embodiments, said CD3 CD561 cells in said NK cell population comprises CD3 CD561 cells that are additionally CD161+. In certain embodiments, said CD3 CD561 cells in said NK cell population comprises CD3 CD561 cells that are additionally DNAM-1+. In certain embodiments, said CD3 CD561 cells in said NK cell population comprises CD3 CD561 cells that are additionally T-bet+.

In one embodiment, an NK cell population produced by a three-stage method described herein comprises cells which are CD117+. In one embodiment, an NK cell population produced by a three-stage method described herein comprises cells which are NKG2D+. In one embodiment, an NK cell population produced by a three-stage method described herein comprises cells which are NKp44+. In one embodiment, an NK cell population produced by a three-stage method described herein comprises cells which are CD244+. In one embodiment, an NK cell population produced by a three-stage method described herein comprises cells which express perforin. In one embodiment, an NK cell population produced by a three-stage method described herein comprises cells which express EOMES. In one embodiment, an NK cell population produced by a three-stage method described herein comprises cells which express granzyme B. In one embodiment, an NK cell population produced by a three-stage method described herein comprises cells

The present disclosure may be better understood by reference to the following non-limiting examples, which are provided as exemplary of the disclosure. The following examples are presented in order to more fully illustrate the preferred embodiments of the disclosure. They should in no way be construed, however, as limiting the broad scope of the disclosure.

### Examples

Natural killer (NK) cells, a crucial component of the innate immune system, are of significant clinical interest for their anti-tumor properties. NK cells can display antibody dependent cellular cytotoxicity (ADCC) activity against tumor cells via the CD16 Fc receptor in combination with tumor specific antibodies. IL-15 is a pleiotropic cytokine that plays a critical role in the survival, proliferation, and cytotoxicity of NK cells. Disclosed herein are human placental CD34+-derived, cryopreserved, off-the-shelf, allogeneic NK cells (CYNK-201) transduced with high IgG binding affinity, protease cleavage resistant CD16 and secreted IL-15 for the treatment of solid and liquid tumors. Here we report both in vitro efficacy results of CYNK-201 against HER2+ gastric cancer NCI-N87 and CD20+ Burkitts lymphoma Daudi cells and in vivo persistence, biodistribution and anti-tumor activity of CYNK-201 in a gastric tumor model.

### Methods and Materials

Cell culture: Human placental CD34+ cells were transduced with a lentivirus vector expressing the CD16 variant and secreted IL-15 and cultured with cytokines to generate CYNK cells.

Cell characterization: Upon completion of cell expansion and differentiation, CYNK cells were frozen. All subsequent characterization and functional analyses were performed on thawed product. CYNK cells were evaluated for surface markers CD56, CD3, CD16, CD226, NKG2D, CD94, CD11a, NKp30, NKp44 & NKp46 by flow cytometry immediately post thaw.

Evaluation of CYNK in vitro anti-tumor activity in combination with antibodies: CYNK cells against HER2+ gastric cancer NCI-N87 cells were assessed with 1 μg/mL Trastuzumab (Genentech) or the IgG control antibody at various E:T ratios. Similarly, CYNK cells against CD20+ Burkitts lymphoma Daudi cells were assessed in combination with 0.1 μg/ml Rituximab (Genentech). ADCC activity was measured by real-time xCELLigence assay (ACEA Biosciences) and cytokine secretion by Luminex xMAP multiplex assay (Millipore Sigma).

Secreted IL-15 from CYNK-201 was measured by a human IL-15 ELISA kit (R&D Systems) on the day of harvest.

In vivo persistence & biodistribution study: CYNK cells were intravenously (IV) injected to NSG mice upon lymphodepletion, with or without recombinant human IL-15 supplementation. 7- or 14-days post injection organs and tissues were collected for digital PCR analysis to detect CYNK cells.

In vivo anti-tumor activity study: CYNK cells were IV injected into NSG mice with NCI-N87 tumor cell inoculation and lymphodepletion. Mice treated with vehicle, trastuzumab alone, or antibody plus CYNK cells were compared. Tumor growth was monitored by measuring volumes.

GraphPad Prism 9.3.1 (GraphPad Prism Software, Inc.) was used to calculate statistics. Data are presented as mean±SEM.

### Results

### CYNK-201 Phenotype & Characterization

>93% NK (CD56+CD3−) purity was achieved.

50.7%-83.5% CD16 expression was maintained post thaw, and the cleavage resistance of CD16 was demonstrated post PMAi stimulation.

IL-15 secretion of CYNK-201 was confirmed.

### CYNK-201 In Vitro Functionality

CYNK-201 cells displayed enhanced trastuzumab or rituximab-mediated ADCC activity against HER2⁺ gastric NCI-N87 or CD20⁺ lymphoma Daudi cells.

CYNK-201 cytokine secretion profile is also consistent with its improved functionality upon combination of trastuzumab.

### CYNK-201 In Vivo Persistence & Anti-Tumor Activity

Compared to CYNK-NT cells, CYNK-201 showed similar biodistribution pattern yet significantly enhanced persistence. CYNK-201 showed superior in vivo ADCC mediated anti-tumor activity in the gastric tumor model.

### CONCLUSION

### CYNK-201 Phenotype & Characterization

>93% NK (CD56+CD3−) purity was achieved. 50.7%-83.5% CD16 expression was maintained post thaw, and the cleavage resistance of CD16 was demonstrated post PMAi stimulation. IL-15 secretion of CYNK-201 was confirmed.

### CYNK-201 In Vitro Functionality

CYNK-201 cells displayed enhanced trastuzumab or rituximab-mediated ADCC activity against HER2⁺ gastric NCI-N87 or CD20⁺ lymphoma Daudi cells. CYNK-201 cytokine secretion profile is also consistent with its improved functionality upon combination of trastuzumab.

### CYNK-201 In Vivo Persistence & Anti-Tumor Activity

Compared to CYNK-NT cells, CYNK-201 showed similar biodistribution pattern yet significantly enhanced persistence. CYNK-201 showed superior in vivo ADCC mediated anti-tumor activity in the gastric tumor model.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A placental derived natural killer (NK) cell or population thereof wherein the placental derived NK cell comprises, or one or more cells within the population of the placental derived NK Cell comprises:
(a) a first polynucleotide encoding a cleavage resistant CD 16 polypeptide and a second polynucleotide encoding a secreted IL-15 polypeptide; or
(b) a polynucleotide that encodes the cleavage resistant CD16 polypeptide and the secreted IL-15 polypeptide.

2. The cell or population of claim 1, wherein the polynucleotide construct further comprises a polynucleotide that encodes a self-cleaving peptide.

3. The cell population of claim 2, wherein the self-cleaving protein is a T2A peptide.

4. The cell or population of cells of claim 2, wherein the polynucleotide construct encodes a polypeptide comprising the amino acid sequences of the cleavage resistant CD16, the T2A protein, and the secreted IL-15, wherein the cleavage resistant CD16 variant comprises a Valine residue at position 176 relative to the wild-type CD 16 polypeptide.

5. The cell or population of cells of claim 4, wherein the polypeptide comprises the amino acid sequence of SEQ ID NO:7.

6. The cell or population of cells of claim 1, wherein the CD 16 is selected from the group consisting of a CD 16a isoform and a CD 16b isoform.

7. The cell or population of cells of claim 6, wherein the CD16b isoform is selected from the group consisting of an NA1 allelic variant and an NA2 allelic variant.

8. The cell or population of cells of claim 1, wherein the cleavage resistant CD 16 variant comprises a residue other than serine or proline at position 197 relative to the wild-type CD 16 polypeptide.

9. The cell or population of cells of claim 1, wherein the cleavage resistant CD 16 variant comprises an amino acid sequence comprising:
(a)
[SEQ ID NO: 1]
TTTPAPRPPTPAPTIASQPLSLRPEACRPAAGGAVHTRGLDFACD
or
at least 15 consecutive residues of [SEQ ID NO: 1];
(b)
[SEQ ID NO: 2]
IEVMYPPPYLDNEKSNGTIIHVKGKHLCPSPLFPGPSKP,
or
at least 15 consecutive residues of [SEQ ID NO: 2];
(c)
[SEQ ID NO: 3]
PELELQVLGLQLPTPVWFH,
or
at least 15 consecutive amino acids of the sequence
PELELQVLGLQLPTPVWFH [SEQ ID NO: 3].

10. The cell or population of cells of claim 1, wherein the cleavage resistant CD 16 variant comprises an amino acid selected from the group consisting of:
[SEQ ID NO: 4]
MWQLLLPTALLLLVSAGMRTEDLPKAVVFLEPQWYRVLEKDSVTLKCQG
AYSPEDNSTQWFHNESLISSQASSYFIDAATVDDSGEYRCQTNLSTLSD
PVQLEVHIGWLLLQAPRWVFKEEDPIHLRCHSWKNTALHKVTYLQNGKG
RKYFHHNSDFYIPKATLKDSGSYFCRGLVGSKNVSSETVNITITQGLAV
CTISSFFPPGYQVSFCLVMVLLFAVDTGLYFS VKTNIRS
STRDWKDHKFKWRKDPQDK.

11. The placental derived NK Cell or population of claim 1, wherein the placental derived NK T-cells are placental CD34+ cell-derived (NK) cells.

12. The placental derived NK Cell or population thereof of claim 5, wherein the polynucleotide construct encodes the amino acid sequence of SEQ ID NO:7.

13. The placental derived NK cell or population thereof of claim 12, wherein the polynucleotide construct comprises the nucleotide sequence of:
[SEQ ID NO: 8]
ATGTGGCAGCTGCTCCTCCCAACTGCTCTGCTACTTCTAGTTTCAGCTG
GCATGCGGACCGAGGACCTGCCCAAGGCCGTGGTGTTCCTGGAGCCCCA
GTGGTACCGGGTGCTGGAGAAGGACAGCGTGACCCTGAAGTGCCAGGGC
GCCTACAGCCCCGAGGACAACAGCACCCAGTGGTTCCACAACGAGAGCC
TGATCAGCAGCCAGGCCAGCAGCTACTTCATCGACGCCGCCACCGTGGA
CGACAGCGGCGAGTACCGGTGCCAGACCAACCTGAGCACCCTGAGCGAC
CCCGTGCAGCTGGAGGTGCACATCGGCTGGCTGCTGCTGCAGGCCCCCC
GGTGGGTGTTCAAGGAGGAGGACCCCATCCACCTGCGGTGCCACAGCTG
GAAGAACACCGCCCTGCACAAGGTGACCTACCTGCAGAACGGCAAGGGC
CGGAAGTACTTCCACCACAACAGCGACTTCTACATCCCCAAGGCCACCC
TGAAGGACAGCGGCAGCTACTTCTGCCGGGGCCTGGTGGGCAGCAAGAA
CGTGAGCAGCGAGACCGTGAACATCACCATCACCCAGGGCGTGATCACC
GCCCTGAGCAGCAGCTTCTTCCCCCCCGGCTACCAGGTGAGCTTCTGCC
TGGTGATGGTGCTGCTGTTCGCCGTGGACACCGGCCTGTACTTCAGCGT
GAAGACCAACATCCGGAGCAGCACCCGGGACTGGAAGGACCACAAGTTC
AAGTGGCGGAAGGACCCCCAGGACAAGGGAAGTGGTGAAGGACGGGGAT
CACTGCTCACTTGCGGGGACGTCGAAGAGAACCCAGGCCCAATGCGAAT
TTCTAAGCCTCACCTCCGAAGTATTAGCATACAGTGCTATCTGTGTCTT
TTGCTGAACTCTCACTTTCTGACCGAAGCGGGAATCCATGTATTCATCC
TCGGGTGTTTTTCAGCGGGCCTCCCAAAGACAGAGGCGAACTGGGTCAA
CGTCATATCTGACCTCAAGAAAATTGAGGATTTGATACAATCAATGCAT
ATTGATGCGACACTCTACACTGAGAGCGATGTTCACCCAAGCTGTAAGG
TAACGGCTATGAAATGTTTTCTCTTGGAGCTCCAGGTTATTTCATTGGA
GTCAGGGGACGCAAGTATACACGACACTGTGGAAAATCTTATAATCCTG
GCTAATAACTCCCTCAGTTCTAATGGAAATGTCACTGAGTCTGGATGCA
AAGAGTGCGAAGAGTTGGAAGAGAAGAATATCAAAGAGTTTCTTCAGTC
CTTCGTGCATATAGTCCAAATGTTTATCAACACCTCCTGATAA;
or
(ii) a degenerate variant thereof.

14. A vector comprising:
(a) a first polynucleotide encoding a cleavage resistant CD 16 polypeptide operatively associated with a promoter;
(b) a second polynucleotide encoding a secreted IL-15 polypeptide operatively associated with a promoter; or
(c) a polynucleotide construct that ecodes a polypeptide comprising:
(i) the amino acid sequence of a cleavage resistant CD16,
(ii) the amino acid sequence of a T2A polypeptide, and
(iii) the amino acid sequence of a secreted IL-15 polypeptide.

15. The vector of claim 14, wherein the vector is a lentivirus vector.

16. The vector of claim 14, comprising the polynucleotide construct of SEQ ID NO:7 operatively associated with a promoter.

17. A placental derived natural killer (NK) Cell or population thereof wherein the placental derived NK cell comprises, or one or more cells within the population of the placental derived are transformed or transfected with the vector of claim 16.

18. A method of suppressing the proliferation of tumor cells comprising contacting the tumor cells with
(i) a placental derived natural killer (NK) cell or one or more cells of population thereof of claim 17; and
(ii) a cancer specific antigen antibody.

19. The method of claim 18, the cancer specific antigen antibody comprises:
(a) a human cancer specific antigen antibody;
(b) a humanized cancer specific antigen antibody;
(c) a chimeric cancer specific antigen antibody;
(d) a monoclonal cancer specific antigen antibody;
(e) a polyclonal cancer specific antigen antibody; or
(f) any combination of (a)-(e).

20. The method of claim 19, wherein the cancer specific antigen antibody comprises trastuzumab ([HERCEPTIN]), or rituximab ([RITUXAN]).

21. The method of claim 18, wherein the tumor cells are CD20+ lymphoma tumor cells.


SEQUENCE LISTING (8 sequences)

SEQ ID NO: 1 - protein, 45 aa, synthetic construct
TTTPAPRPPTPAPTIASQPLSLRPEACRPAAGGAVHTRGLDFACD

SEQ ID NO: 2 - protein, 39 aa, synthetic construct
IEVMYPPPYLDNEKSNGTIIHVKGKHLCPSPLFPGPSKP

SEQ ID NO: 3 - protein, 16 aa, synthetic construct
PELELQVLGLQLPTPV

SEQ ID NO: 4 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVCTISSFFPPGYQVSFCLVMVLLFAVDTGLYFSVKTNIRSSTRDWKDHKFKWRKDPQDK

SEQ ID NO: 5 - protein, 6 aa, Synthetic construct
HHHHHH

SEQ ID NO: 6 - protein, 10 aa, synthetic construct
EQKLISEEDL

SEQ ID NO: 7 - protein, 435 aa, synthetic construct
WQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGVITALSSSFFPPGYQVSFCLVMVLLFAVDTGLYFSVKTNIRSSTRDWKDHKFKWRKDPQDKGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 8 - DNA, 1317 bp, Synthetic construct
  source 1..1317: mol_type = other DNA
atgtggcagctgctcctcccaactgctctgctacttctagtttcagctggcatgcggaccgaggacctgcccaaggccgtggtgttcctggagccccagtggtaccgggtgctggagaaggacagcgtgaccctgaagtgccagggcgcctacagccccgaggacaacagcacccagtggttccacaacgagagcctgatcagcagccaggccagcagctacttcatcgacgccgccaccgtggacgacagcggcgagtaccggtgccagaccaacctgagcaccctgagcgaccccgtgcagctggaggtgcacatcggctggctgctgctgcaggccccccggtgggtgttcaaggaggaggaccccatccacctgcggtgccacagctggaagaacaccgccctgcacaaggtgacctacctgcagaacggcaagggccggaagtacttccaccacaacagcgacttctacatccccaaggccaccctgaaggacagcggcagctacttctgccggggcctggtgggcagcaagaacgtgagcagcgagaccgtgaacatcaccatcacccagggcgtgatcaccgccctgagcagcagcttcttcccccccggctaccaggtgagcttctgcctggtgatggtgctgctgttcgccgtggacaccggcctgtacttcagcgtgaagaccaacatccggagcagcacccgggactggaaggaccacaagttcaagtggcggaaggacccccaggacaagggaagtggtgaaggacggggatcactgctcacttgcggggacgtcgaagagaacccaggcccaatgcgaatttctaagcctcacctccgaagtattagcatacagtgctatctgtgtcttttgctgaactctcactttctgaccgaagcgggaatccatgtattcatcctcgggtgtttttcagcgggcctcccaaagacagaggcgaactgggtcaacgtcatatctgacctcaagaaaattgaggatttgatacaatcaatgcatattgatgcgacactctacactgagagcgatgttcacccaagctgtaaggtaacggctatgaaatgttttctcttggagctccaggttatttcattggagtcaggggacgcaagtatacacgacactgtggaaaatcttataatcctggctaataactccctcagttctaatggaaatgtcactgagtctggatgcaaagagtgcgaagagttggaagagaagaatatcaaagagtttcttcagtccttcgtgcatatagtccaaatgtttatcaacacctcctgataa